SEQUENCE LISTING (16 sequences)

SEQ ID NO: 1 - DNA, 59 bp, Artificial Sequence
  oligonucleotide primer
cgcccgccgcgccccgcgcccgtcccgccgcccccgcccgctggtgggtttggttttgg

SEQ ID NO: 2 - DNA, 22 bp, Artificial Sequence
  oligonucleotide primer
gagcagagggagacagagagag

SEQ ID NO: 3 - DNA, 60 bp, Artificial Sequence
  oligonucleotide primer
cgggcgggggcggcgggacgggcgcggggcgcggcgggcgagcatttactgcagcttgct

SEQ ID NO: 4 - DNA, 20 bp, Artificial Sequence
  oligonucleotide primer
acctcctcaaacagctcaaa

SEQ ID NO: 5 - DNA, 60 bp, Artificial Sequence
  oligonucleotide primer
cgcccgccgcgccccgcgcccgtcccgccgcccccgcccgaaggtatgtgcattgttttt

SEQ ID NO: 6 - DNA, 21 bp, Artificial Sequence
  oligonucleotide primer
ccgcaataaagcaaatattac

SEQ ID NO: 7 - DNA, 62 bp, Artificial Sequence
  oligonucleotide primer
cgcccgccgcgccccgcgcccgtcccgccgcccccgcccgcctctattgttggatcatattc

SEQ ID NO: 8 - DNA, 20 bp, Artificial Sequence
  oligonucleotide primer
cgcccgccgcgccccgcgcc

SEQ ID NO: 9 - DNA, 21 bp, Artificial Sequence
  oligonucleotide primer
atgactgaatataaacttgtg

SEQ ID NO: 10 - DNA, 61 bp, Artificial Sequence
  oligonucleotide primer
cgcccgccgcgccccgcgcccgtcccgccgcccccgcccgctatctttgtcagcattgcat

SEQ ID NO: 11 - DNA, 60 bp, Artificial Sequence
  oligonucleotide primer
cgcccgccgcgccccgcgcccgtcccgccgcccccgcccgcttcctgaagacctgaacac

SEQ ID NO: 12 - DNA, 20 bp, Artificial Sequence
  oligonucleotide primer
cagggatgaagagaagaaaa

SEQ ID NO: 13 - DNA, 61 bp, Artificial Sequence
  oligonucleotide primer
gcgggcggcgcggggcgcgggcagggcggcgggggcgggctcaaactattgggtggatttg

SEQ ID NO: 14 - DNA, 22 bp, Artificial Sequence
  oligonucleotide primer
tccctgaacatgtgtagaaagc

SEQ ID NO: 15 - DNA, 20 bp, Artificial Sequence
  oligonucleotide primer
gcgggcggcgcggggcgcgg

SEQ ID NO: 16 - DNA, 20 bp, Artificial Sequence
  oligonucleotide primer
atgctagcaggaagaacaga

(19) United States
(12) Patent Application Publication
(10) Pub. No.: US 2007/0117096 A1
(43) Pub. Date: May 24, 2007

(54) Oscillating temperature capillary electrophoresis and uses therefor

(75) Inventor: Per Olaf Ekstrom, Nesoddtangen (NO)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: THE NORWEGIAN RDIUM HOSPITAL RESEARCH FOUNDATION, Oslo (NO)

(21) Appl. No.: 10/578,937

(22) PCT Filed: Nov. 20, 2003
(86) PCT No.: PCT/IB03/05304

§ 371 Date: Oct. 12, 2006

Related U.S. Application Data
(60) Provisional application No. 60/519,179, filed on Nov. 12, 2003.

(51) Int. Cl. C12Q 1/68

(52) U.S. Cl. 435/6; 204/451

(57) ABSTRACT

Methods for separating biomolecules using oscillating temperature electrophoresis are disclosed. Uses for such methods are also disclosed including high throughput screening, estimation of allele frequencies, identification of polymorphisms, and separation of DNA molecules.

### RELATED APPLICATION

This application claims the benefit of the U.S. Provisional Application No. 60/______, entitled, “Oscillating Temperature Capillary Electrophoresis and Uses Therefor,” by Per Olaf Ekstrøm, filed Nov. 12, 2003, having attorney docket number 3637.1000-000.

The entire teachings of the above application are incorporated herein by reference.

### BACKGROUND OF THE INVENTION

In 1953, Watson and Crick hypothesized the structure of the DNA molecule. Approximately five decades later the draft sequence of the human genome was completed. Decoding of the human DNA sequence through the Human Genome (HUGO) project differences in the genomes were analyzed. The most frequent DNA variations, have elucidated single nucleotide polymorphisms (SNPs), are considered to be useful polymorphic markers for genetic studies of pharmacogenetics and polygenic traits. A worldwide effort has achieved an accumulation of millions of SNPs stored in public databases. However, most of these SNPs have been identified by examination of a limited number of individuals, and information on their allele frequencies is lacking or tentative. Furthermore, studies have shown that allele frequencies vary widely between different ethnic populations. Thus validation of SNPs and estimation of their allele frequencies, especially for each ethnic group, are required before these markers can be used for genetic studies.

Current efforts in discovery and screening of DNA variations (point mutations, SNPs and other polymorphisms) are expected to yield valuable information on potential genetic risk factors as well as information on genetic variants that could lead to enhanced susceptibility for certain diseases. It is expected that discovery and massive screening of DNA polymorphisms will become essential for taylor-made drugs as well as disease gene association studies. Screening for and identification of SNPs and other polymorphisms will therefore require methods for separating variant DNA sequences from large samples.

### SUMMARY OF THE INVENTION

Described herein is a new type of DNA variation (polymorphism, mutation or SNP) screening technology based on capillary array electrophoresis. Sample DNA fragments of known sequence are PCR amplified and detected based on their differential migration in a polymer-filled capillary. A cycling (oscillating) temperature is used to compensate for local fluctuations in temperatures across the multi-capillary array. In addition, the application of short periodic temperature cycles allows a user to continue injecting subsequent sample plates between the temperature gradient cycles before the earlier samples appear in the detector. Using this novel approach, a dramatic and unexpected increase in separation throughput by more effective utilization of the separation capacity (volume) of each capillary is demonstrated.

In one embodiment, the present invention is directed to a method for separating nucleic acids comprising electrophoresing a sample applied to a gel electrophoresis matrix in a capillary, wherein during electrophoresis, the temperature of the matrix is cycled at least two times between a high and low temperature. In a particular embodiment, the nucleic acids to be separated are DNA fragments comprising one or more polymorphic sites. In another embodiment, allelic variants at the one or more polymorphic sites are separated. In a particular embodiment, the temperature is initially at a high temperature and the first cycle is from a high temperature to a low temperature. In another embodiment, the high temperature and/or low temperature is different during successive cycles. In another embodiment, the temperature is cycled from about 2 to 60 times. In a particular embodiment, the temperature is cycled about 20 times. In another embodiment, the high temperature is about 3° C. higher than the low temperature. In another embodiment, the temperature is between about 2° C. and about 15° C. higher than the lower temperature. In another embodiment, the higher temperature is between about 3° C. and about 10° C. higher than the lower temperature. In one embodiment, the high temperature is less than about 80° C. In a particular embodiment, the low temperature is about 40° C. In one embodiment, the high temperature is between 50° C. and 75° C. In another embodiment, the low temperature is between 40° C. and 50° C. In a particular embodiment, the method further comprises detecting dsDNA after electrophoresis. In a particular embodiment, after the desired number of temperature cycles have been completed, the temperature of the gel matrix is such that DNA remains double-stranded. In another embodiment, the temperature oscillations are ramped to provide optimal separation of the alleles.

In another embodiment, the present invention,is directed to a method for estimating allele frequency comprising: electrophoresing a sample applied to a capillary gel electrophoresis matrix, wherein during electrophoresis, the temperature of the matrix is cycled at least two times, wherein one cycle is from a high temperature to a low temperature or from a low temperature to a high temperature, thereby separating DNA molecules in the sample; and quantifying the variant sequences of the separated DNA molecules thereby providing an estimate of the allele frequency for each variant DNA molecule. In a particular embodiment, the method further comprises detecting dsDNA after electrophoresis. In one embodiment, after the desired number of temperature cycles have been completed, the temperature of the gel matrix is such that DNA remains double-stranded.

In yet another embodiment, the invention is directed to a method for detecting a microhaplotype comprising separating DNA fragments comprising a sequence comprising two or more polymorphic sites of the microhaplotype, wherein the fragments are separated by capillary electrophoresis performed with two or more temperature oscillations between a high and a low temperature. In a particular embodiment, the temperature oscillations are ramped to provide optimal separation of the microhaplotype.

### BRIEF DESCRIPTION OF THE DRAWINGS

FIGS. 1A-1C are graphs showing temperature over time during different CE methods. FIG. 1A shows multiple-injection CE. FIG. 1B shows multiple- injection TGCE. FIG. 1C shows multiple-injection CGCE. The upper drawing illustrates the temperature profile. The simulated positions of separated sample zones are at the bottom.

FIG. 2 is a spectral profile showing a comparison of a two temperature profile mode. Single samples analyzed for single nucleotide polymorphisms in the LTA gene by temperature gradient capillary electrophoresis. Genotypes of the sample are heterozygous GA; thus four peaks are identified as GC, AT, AC and GT, respectively. Lower part of the figure display programmed temperature profile of the analysis chamber during electrophoresis.

FIGS. 3A and 3B show experimental measurements of temperature profiles inside MegaBACE™ 1000 capillary chamber. FIG. 3A depicts the typical profile of a single-sweep gradient mode. FIG. 3B shows a cycling temperature gradient mode.

The MegaBACE™ 1000 used here was a modified “HOT” MegaBACE™ at Molecular Dynamics. The standard temperature limit, 50° C., on the MegaBACE™ was raised to 65° C.

FIG. 4 is a fluorescence and temperature profile depicting the analysis of several samples for APC mutation detection using multiple-injections and fast cycling temperature gradient conditions. The three injections were performed every fourth temperature cycle. First injection is C homozygote, second injection is T homozygote and the last injection is C/T heterozygote.

FIG. 5 is an illustration of high throughput SNP scoring by CTCE in multi-capillary format. Five different sample plates (FAM-labeled PCR fragments of BRCA2) were consecutively injected during a cycling temperature gradient. Allele scoring was performed against a TMR-labeled internal mutant showing heterozygote profile with both allele peaks. The internal standard was also used as an “injection marker” for consecutive data processing by SNP Profiler software.

FIG. 6 is a schematic diagram depicting an overview of CTCE sample processing workflow.

FIG. 7 is a chart showing the relative thermodynamic stability of mutations as compared to wild-type fragments. Wild-type KRAS exon 1 with the sequence GGT and GGC in codon 12 and 13, respectively, is coded as 100% and the scale indicates relative difference in the thermodynamics profile of the mutants. All G to C mutations proved to be more stable than wild-type, and will thus separate out before wild-type peaks in electropherograms, except for a GGC to GCC mutation that was only slightly more stable than wild-type, and will comigrate with wild-type. All other mutations were less stable than wild-type and will separate after wild-type peaks. Note that some of the mutations had very similar thermodynamics.

FIG. 8 is a representation of electropherograms of all oncogenic mutations in KRAS exon 1 and standard analyzed with CTCE. Separation between the wild-type homoduplex and the heterozygous peaks was achieved in all mutated samples. Separation between the wild-type homoduplex and mutant homoduplex was found in 11 out of 12 mutants. The mutant homozygous peak of GGC to GCC transversion in codon 13 comigrated with wild-type at the selected denaturing conditions. This observation corresponded with the theoretical melting properties of the GCC mutation as compared to wild-type sequence. The thin line represents the sample and the bold line the standard. The peaks of the standard are named S1-S5 from left to right (S1, mutant homoduplex from CGT; S2, mutant homoduplex from TGT; S3, mutant homoduplex from AGC; S4, mutant heteroduplexes from TGT and CGT; S5, mutant heteroduplex from AGC). The peaks of the sample are named WT (wild-type), M (mutant), H1 (heteroduplex with shortest migration time) and H2 (heteroduplex with longest migration time). Note that some of the samples contain a single-strand peak produced under the PCR amplification or under the CTCE analysis.

FIG. 9 is a diagram showing the Genetic Profiler analyzed migration times of all peaks in the electropherograms, and the relative migration times between peaks were calculated by Excel. Individual mutations proved to have distinct migration times when compared to the peaks of the standard and other mutations. Migration times that were able to separate different mutations were selected and a classification tree was made. For example, a TGT mutation was characterized first by a shorter migration time of H1 than S4, and migration time of S4 minus migration time of H1 was less than 12. Finally, the migration time of the mutant homoduplex (M) was longer than for the wild-type peak (WT). No other mutations had these characteristics.

FIG. 10 is a graph showing electropherograms of 5 samples heterozygous in TNF target single-nucleotide polymorphism (SNP). Ratio between the G and A alleles was calculated by measuring the area under the peaks and found to be 0.996 (one SD=0.028).

FIG. 11 is a graph showing electropherograms of the 4 pools representing 2000 alleles each, analyzed for the single-nucleotide polymorphism (SNP) in TNF by denaturant capillary electrophoresis (DCE). The area under the peaks was measured and the allele frequencies calculated. Pool 3 revealed a large single-strand peak (marked X) created either during the PCR or during electrophoresis. The single strand did not interfere with the measurement of peak areas.

FIG. 12 is a table showing allele and genotype frequencies of analyzed SNPs.

FIG. 13 is a plot showing electropherograms of three samples with different genotypes. The samples were analyzed by denaturant capillary electrophoresis (DCE) with internal Tamra-labeled standard (bold line). Upper part shows the homozygous GG genotype, the middle part shows the genotype AA, and lower part shows the heterozygous GA genotype.

FIG. 14 is a plot showing five heterozygous samples injected in 8.5 minute intervals during denaturant capillary electrophoresis (DCE) with a gradient of 54° C. to 50° C. in 1° C. steps. Each temperature was held for 1 minute. The gradient was repeated 8 times.

FIG. 15 A-E are electropherograms from cycling temperature capillary electrophoresis of five genotypes representing the five microhaplotypes combination found. Sample alleles are depicted with thin line and the internal standard are presented as bold line. Microhaplotypes are given as the three polymorphic bases in each allele. F or example, the first peak in the internal standard is IVS38-8 T 5557 G 5558 A, is labeled as TGA. Note that heteroduplexes are not shown in the electropherograms.

FIG. 16 is a table showing the total numbers of haplotypes observed in the normal and rectal cancer groups. Normalized melting behavior of microhaplotypes are presented under calculations. High temperature property is indicated by negative values for the fragments. For positive values, the fragment would be less stable as compared to the wild-type and consequently elute after the wild-type.

FIG. 17 is a table showing the observed genotypes in the normal population (n=3,526) and rectal cancer patients (n=151) and allele frequencies of each SNP.

FIG. 18 is a graph showing relative differences (y-axis) in stability between G and A alleles in lymphotoxin alpha at different melting probabilities (x-axis) are shown. The thermodynamics calculated by the use of the Poland web page (H) predicted the observed stability difference between the alleles (9) at 50% melting probability. WinMelt (F) predicted difference in separation at all probability levels. At 50% melting probability, a good theoretical prediction of observed thermodynamic differences was obtained with both the Poland home page and WinMelt. This level of probability was used for further analyses.

FIG. 19 is a plot showing the correlation between predicted thermodynamics at 50% melting probability with WinMelt and relative migration time in DCE under optimal denaturing conditions. A linear correlation with r²=0.97 was found.

FIG. 20 is a representation showing temperature measured at three different locations with calibrated external temperature sensors in the capillary chamber at three different temperature settings. Temperatures in different parts of the capillary chamber were recorded continuously for a period of 15 min. The dotted line represents the intended temperature in the chamber. Temperatures measured around the capillaries in the front are indicated with an A, the capillaries in the middle with a B, and the capillaries at the back with a C. The upper part of the figure shows the temperature recorded at different locations with a preset constant temperature in the chamber. In the middle part of the figure a gradient going through the theoretical denaturing temperature is employed. In the lower part of the figure, cycling of temperature around the intended temperature is illustrated.

FIG. 21 is a plot showing relative retention of the two homoduplexes and two heteroduplexes in a heterozygous sample analyzed with DCE at different predefined denaturing temperature. One standard deviation is noted in the figure for each homoduplex (n=11) and heteroduplex (n=11).

FIGS. 22A and 22B are plots showing electropherograms. In FIG. 22A, two electropherograms of the same PCR fragment, analyzed in the same run and in differentcapillaries under constant denaturant conditions, are shown. A difference in separation is observed between the two capillaries. In FIG. 22B the same PCR fragments are analyzed with cycling temperature, with a gradient from 48° C. to 52° C. and cycled 15 times. Each temperature was held for 30 s. With these denaturing conditions, equal migration times were observed in different capillaries.

FIG. 23 is a plot showing a KRAS exon 1 internal standard with three homoduplex peaks and two heteroduplex peaks was analyzed with CTCE in 96 capillary format. The peaks were measured in 649 individual samples and presented as box plots to demonstrate the reproducibility in DCE analysis with cycling temperature.

FIG. 24 is a theoretical melting profile of twelve double-stranded DNA fragments as analyzed by the WinMelt computer program. The following DNA sequences are depicted: 1) BRCA 1,2) BRCA 1,3) MTHFR, 4) OPSIN, 5) MTHFR, 6) MTHFR, 7) CBS, 8) NQO1, 9) DPYD, 10) DPYD, 11) DPYD, 12) CTLA-4. Theoretical melting temperature is depicted on the y-axis, and number of base pairs on the x-axis. Note that the maximum difference in thermodynamics between two fragments is 9.4 K.

FIG. 25 is a plot showing electropherograms of all twelve fragments when analyzed with CTCE and cycling denaturant temperature from 59-47° C. and 5 cycles. The fragments are arranged as in FIG. 24. Note the baseline separation between the two homoduplexes and between homoduplexes and heteroduplexes in all electropherograms.

FIG. 26 is a schematic showing the difference in migration times of all peaks after CTCE analysis with cycling denaturant temperature from 59-47° C. and 5 cycles. The results are illustrated as average (n=8) peak maximum with one standard error of the mean.

FIG. 27 is a plot showing temperature measured at three different locations with calibrated external temperature sensors recorded continuously during electrophoresis. The cycling condition was temperatures programmed from 59-47° C. for 5 cycles. Temperature measured in the front, middle and back of the chamber are as indicated in the legend.

FIG. 28 is a graph illustrating the difference in migration time between the peaks of a heterozygous PCR sample at different denaturing conditions. Each point in the graph represent separation at 3 K gradient with average gradient temperature starting at 42.5 C. and increasing by one ° C. Note that separation of individual peaks can be achieved within a quite large temperature frame.

FIG. 29 is a plot showing observed denaturing temperature in the capillary chamber at three different locations. The programmed denaturing temperature was from 47° C. to 55° C. and with 7.5 cycles. Temperature measured in the front, middle and back of the chamber are as indicated in the legend.

FIG. 30 is a plot showing the difference in migration times of all peaks after CTCE analysis with cycling denaturant temperature from 47-55° C. and 7.5 cycles. The results illustrate average peak maximum and one standard error of the mean. Note that fragments with high theoretical thermodynamics (number 6 and 7) have insufficient separation.

FIG. 31 is a plot showing the theoretical thermodynamics of a target sequence. Base substitution A->G in the lower domain changes the melting properties as shown in the enlargement.

FIG. 32 is a plot showing three samples analyzed for mutation in KRAS exon 1. The upper electrophreogram shows the wild-type sample, whereas the two lower samples harbor GGT>GTT mutations. Peaks numbered 1 to 4 represent the wild-type homoduplex, mutant homoduplex and heteroduplexes, respectively. Note the different mutant fraction in the two lower electropherograms.

FIG. 33 is a plot showing analysis of SNP in IL-4 gene by DCE.

Identification of genotypes by co-elution with an internal standard (bold line) made up of both alleles. The genotypes from top to bottom are TT, CT and CC, respectively. Heteroduplexes are not shown.

FIG. 34 is a table showing microhaplotype combination for two SNPs separated by 57 bp.

FIG. 35 is a plot showing five samples microhaplotyped according to an internal standard (bold line). Genotypes are given as allele combination as numbered in FIG. 34. Heteroduplexes are not shown.

FIG. 36 is a plot showing quantitative analysis of gene copy numbers in Opsin gene array. The ratio of L/M based on area under the peaks is 1/3. Note the non-template, polymerase-mediated adenine addition (a), which does not interfere with the measurements.

FIG. 37 is a plot showing allelic imbalance analyzed by DCE. The ratio of the two alleles in the upper sample is 1:1 as expected for a heterozygous sample. In the lower electropherogram a clear imbalance between alleles can be seen.

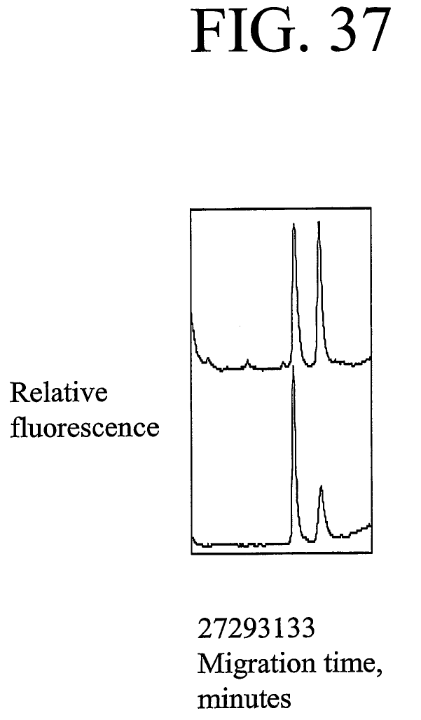

### DETAILED DESCRIPTION OF THE INVENTION

Described herein is an oscillating temperature gradient electrophoresis system useful for separating biomolecules, e.g., DNA. The methods described herein can be used in combination with multiple-injection technology to dramatically increase the sample throughput (Mansfield, E. et al., 2000, U.S. Pat. No. 6,156,178). The present work extends technology for detection of DNA variants into a high throughput mode. Methods for screening multiple samples for the presence of somatic mutations after transferring a high-resolution technique of CDCE from single-capillary format to a commercial capillary array instrument had been described (Bjørheim, J. et al., 2002, Anal. Biochem., 304:200-205). Additional advantages of applying an oscillating temporal temperature gradient rather than maintaining an accurate constant temperature for increased reproducibility in a commercial multi-capillary instrument were later described (Minarik, M. et al., 2001, poster presented at ICHG Conference, Vienna, Austria).

Samples suitable for the present invention can be biological samples obtained from, for example, animals, plants, viruses or bacteria. Animal samples can be, for example, mammalian in origin and derived from, for example, blood, serum, cells tissue. For example, a biological sample from a test subject (a “test sample”) containing a biomolecule of interest, e.g., genomic DNA, RNA, protein, polypeptide or cDNA, is obtained from an individual or several samples can be collected from multiple individuals in a population. The individual can be an adult, child, or fetus. The test sample can be from any source that contains the desired biomolecule, preferably DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling.

Among the various experimental techniques for detection of DNA variations, DNA sequencing is the most universal approach. Although sequencing has been the “gold standard” for detecting polymorphisms for many years, a variety of alternative approaches for detection and screening have emerged, overcoming the high cost and occasional lack of sensitivity. These methods can rely on hybridization, allele-specific enzymatic reactions such as PCR, minisequencing, strand displacement, and/or cleavage, or on differences in physicochemical properties of the DNA variants such as the melting equilibrium, or affinity towards stationary phase. The results of these techniques can be read directly from the reaction mixture using plate readers or following separation of nucleic acids by capillary electrophoresis (CE), HPLC or mass spectrometry. The techniques employing CE, HPLC or mass spectrometry offer an additional means of identification of the variation from a characteristic pattern of migration, elution or mass spectra. With many of the above-mentioned screening techniques, the sample throughput can be increased by processing multiple samples in parallel. This feature becomes useful when confronted with the vast amount of potential sequence targets to be scrutinized.

Electrophoresis has traditionally been used for separation of DNA fragments, DNA sequencing as well as general fragment analysis. A large family of slab-gel techniques dedicated to detection of DNA variations is based on differential melting of wild-type (taken to be a particular reference sequence) and mutant (a sequence that varies from the wild-type sequence) DNA fragments translated into an observable retention difference through electrophoretic sieving. One technique that utilizes this principle is denaturing gradient gel electrophoresis (DGGE), in which amplified fragments of wild-type and mutant sequences are resolved during their migration in a slab gel containing a gradient of chemical denaturant. DGGE is well established in clinical diagnostics due to its relative simplicity and ability to resolve close to 100% of mutations present in a sample for a given target sequence. Following DGGE, other variants of slab-gel mutant separation methods were developed including temporal temperature gradient gel electrophoresis (TTGE), in which the temperature is varied throughout separation and constant denaturing gel electrophoresis (CDGE), where the separation takes place at predetermined constant denaturing conditions.

In addition to the electrophoretic methods, an approach based on denaturing HPLC (dHPLC) has been used to identify polymorphsims. dHPLC uses an ion-pair chromatography separation principle combined with precise control of the column temperature and optimized mobile phase gradient for separation of mutant heteroduplexes. dHPLC can be easily automated and offers an option to collect the isolated heteroduplexes for further identification or confirmation by sequencing. However, the main potential of DGGE and DHPLC is mostly in discovery of novel mutations rather than screening due to their relatively low throughput of approximately 5 min per analysis.

The transition in DNA separations from traditional slab-gel electrophoresis to CE systems started in early 1990 and was later further accelerated by the Human Genome Project. Separation of heteroduplexes was achieved at different temperatures controlled by means of Joule heating through adjustment of the separation voltage. In 1994, a method referred to as constant denaturing capillary electrophoresis (CDCE) was introduced as a capillary analog to CDGE, a constant denaturing electrophoresis performed on slab gels. For CDCE, the separation is carried out at an accurately maintained constant temperature at which the homo- and heteroduplex forms of wild-type and mutant sequences exhibit the best separation. An alternative approach was later used that overcomes the requirement for a very accurate temperature control by applying a simple temporal temperature gradient. Applying a temperature gradient, rather than a constant temperature, is useful especially in multi-capillary systems where maintaining accurate temperature across all capillaries is difficult. With a temperature gradient, each capillary reaches its temperature optimum, even if there is a difference in absolute temperature values among capillaries. The duration of such optimum separation conditions only depends on the overall slope of the temperature gradient.

The temperature gradient approach can be applied to many existing commercial multi-capillary CE systems with no additional requirements on instrument hardware. The typical run time is less than 60 minutes, depending on the resolution requirement (i.e., gradient slope).

The present invention encompasses methods to improve separation of, for example, different alleles of a DNA fragment. An “allele” is a particular sequence version present at a “polymorphic site”, i.e., a site that, in a particular population of molecules, can have more than one nucleotide present. For example, a specific position of a DNA fragment of a human chromosome might have an adenine in one molecule and a cytidine in another molecule at the same position. The position would be a “polymorphic site” having at least two possible alleles: the “A” allele and the “C” allele. Since the fragments might otherwise be identical in sequence (see Example 7 for a discussion of “microhaplotypes”), the problem of separating the A allele from the C allele is significant. Described herein are CE methods for separating DNA fragments that might differ in sequence at only one position. Samples are PCR-amplified to attach a “GC-clamp” (a sequence high in guanine and cytidine residues), and subsequently separated by Capillary electrophoresis. By allowing the temperature to oscillate during the electrophoresis run, it was unexpectedly observed that separation of biomolecules was dramatically improved.

The methods of the present invention can be used in conjunction with known and yet to be discovered electrophoresis methods to improve, for example, throughput. For example, temperature oscillations can be introduced during multiple injection capillary electrophoresis to further improve resolution and increase the amount of sample that can be separated during a given time period. Additionally, the invention can be used in parallel in multi-capillary systems.

In a multiple-injection experiment, samples are serially injected into the capillary (or array of capillaries) in periodical time intervals separated by short application of separation voltage. This approach is an efficient usage of the separation capacity (migration volume) of the capillary column. Following injection of the first sample (or a set of samples in capillary array), a so-called interval voltage is applied for a sufficient period of time (typically 2-5 min for short oligonucleotides) preventing overlap of the slowest peak from the sample with the fastest migrating peak from the next sample. After this period, the next sample is injected resulting in a continuing process during which the first set of samples is reaching detectors, the following set is separating and new ones are being injected. In the most common version, the injections are repeated several times before the fastest migrating peaks from the first sample reach the detector. After the final injection, a run voltage is applied for a longer period of time (30-60 minutes) to drive all peaks from all injections pass the detection window. The maximum number of injections are determined experimentally.

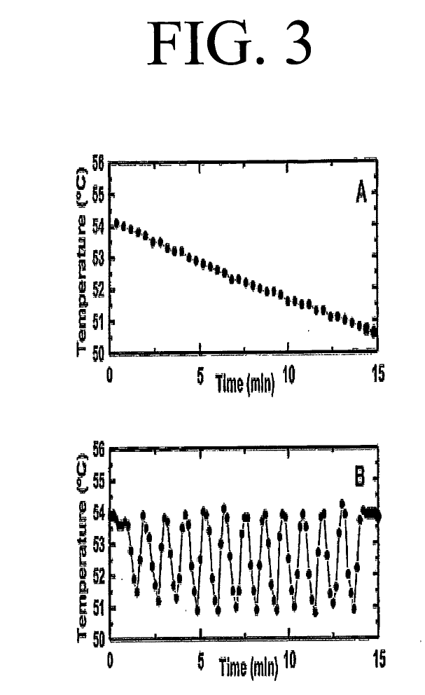

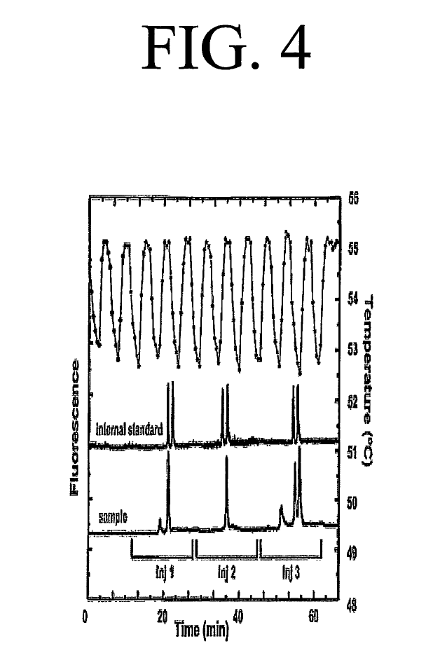

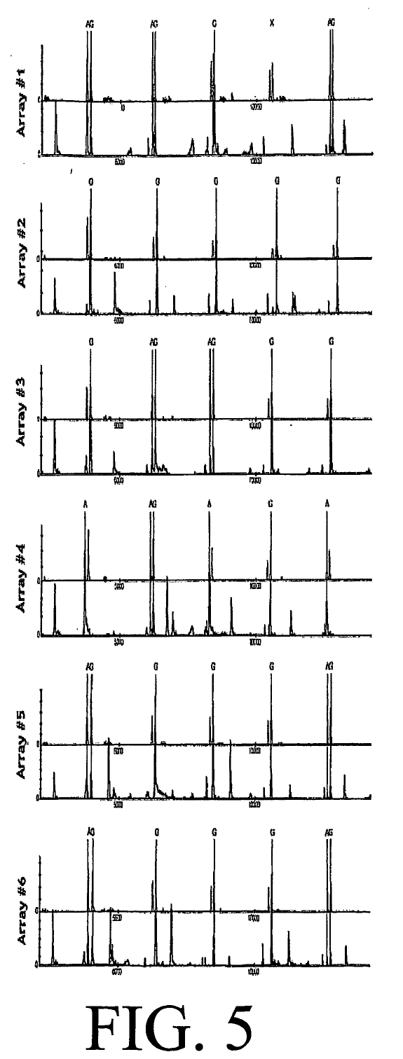

Most commercial capillary array instruments allow controlling temperature during the run. FIG. 3 shows the temperature profile of a single-sweep gradient (A) and atypical cycling gradient (B) recorded inside the capillary chamber of MegaBACE 1000 capillary array instrument equipped with high temperature setting. The unchanged separation performance of the cycling gradient compared to a single-sweep gradient allows for the application of the multiple-injection method. Depending on the actual application, the samples can be periodically injected either in every gradient cycle or once in every few cycles (e.g., every cycle, every 2 cycles, every 3 cycles, every 4 cycles, every 5 to 10 cycles, every 7 to 20 cycles, every 15 to 40 cycles, etc.). A single cycle can start at either high temperature limit or the low temperature limit. An example of multiple-injection cycling temperature gradient capillary electrophoresis (CGCE) with fast temperature cycling (5 minutes per cycle) is shown in FIG. 4. APC mutant samples were injected in every fourth gradient cycle. During each cycle, a temperature gradient going from 55° C. to 53° C. and back to 55° C. was applied. An internal standard containing 1:1 mixture of individually amplified wild-type and mutant fragments labeled with a TMR dye was included in each sample well. Since no heteroduplexes were formed following the PCR, the resulting separation patterns consist only of homoduplexes. It can be seen that a full separation of both wild-type and mutant homoduplexes of the internal standard was achieved within the individual interval windows. Clearly, a very high resolution of the peak separation is achieved under the cycling temperature gradient conditions, allowing identification of both alleles in a heterozygous sample by comparison to the peak pattern of the standard. Apart from just detecting a presence of DNA variation from the characteristic peak pattern in heterozygous samples, the complete separation of the two homoduplexes allows direct identification of homozygous genotypes. This is the key in automated SNP genotyping, where the two homozygous genotypes can be directly scored. An example of high-throughput analysis and automated scoring of SNPs is shown in FIG. 5. A BRCA2 SNP was screened in various patients using the five injections with 45° C.-43° C. cycling temperature gradient. Each sample included a 6-carboxy-N,N,N′,N′-tetramethylrhodamine (TAMRA)-labeled internal standard. The data was processed using SNP Profiler software which allows assignment of individual injection windows. The genotypes are directly determined from the positions of mutant homoduplex peaks co-eluting with the internal standard homoduplex peaks.

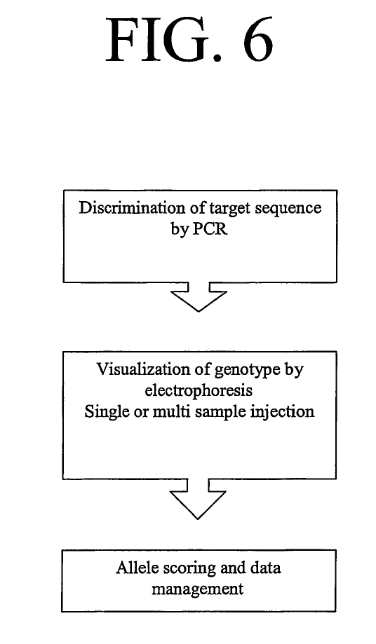

The total runtime of the experiment shown in FIG. 5 was less than 2 hours. It was estimated that, on a common 96-capillary instrument, five injections could feasibly be performed without any adverse effect on the separation matrix. The total runtime would then be 20 min of initial “dead” volume+5×15 minutes separation window+15 minutes final electrophoresis=110 min. Considering 10 minute additional periods required for gel replacement in between runs, a total of 12 of similar multi-injection runs can be performed in less than 24 hours of operation. This represents an overall throughput of 5760 samples in 24 hours. Commercial genetic analyzers are usually equipped with four dye channels. Considering that up to three fluorescent dye channels can be used to detect unknown samples (the last channel is assigned for the internal standard), it is clear that the potential capacity can be further increased 3-fold up to 17,200 samples in 24 hours on a single 96-capillary instrument. Unlike in other mutant or SNP scoring methods, the presented technology includes a very straightforward workflow shown in FIG. 6. Following the original PCR amplification, there is no sample cleanup required. Using a 96-format, PCR thermocycler enables complete automation from sample preparation to the multiple-injection CE analysis using robotic plate handlers.

The methods of the present invention can be used in conjunction for separating biomolecules (DNA, RNA, DNA:RNA hybrids, polypeptides, proteins, carbohydrates, etc.) that are conventionally separated by electrophoresis. The methods described herein apply to those biomolecules that are affected by temperature variation during separation. The method is not limited to CE, but can be applied to other electrophoresis methods as well. For example, temperature oscillations can be introduced during slab gel electrophoresis, polyacrylamide gel electrophoresis (AGE), sodium dodecyl sulfate PAGE, etc.

The temperature oscillations can be performed with a high temperature boundary approximating the temperature at which the GC-clamp melts. For example, a GC-clamp having a melting temperature of 85° C. will have a practical high temperature limit of 85° C. during electrophoresis. The practical lower temperature limit for temperature oscillations during electrophoresis is the minimal melting temperature of AT-rich DNA sequences. In practical terms, the lower temperature limit is about 35° C. Thus, temperature oscillations can be performed from about 35° C. to about 85° C., depending on the fragment to be separated and the resolution desired. The difference between the high and the low temperature limits for the oscillations can be, for example, about 1° C., about 2° C., about 3° C., about 5° C., about 7° C., about 10° C., about 15° C., about 20° C., about 25° C., about 30° C., about 35° C., about 40° C., about 45° C. or about 50° C. Additionally, the high and/or low temperature limits for the oscillations can vary from cycle to cycle. For example, the first cycle can have a high temperature T_{H1} and a low temperature T_{L1}, and have a different high temperature during the second cycle, T_{H2} and T_{L2}. Each cycle can have a different high and low temperature, or they can be the same for the purposes of the present invention.

One of skill in the art would know how to vary electrophoresis conditions, e.g., through the addition of compounds such as, for example, urea, such that DNA melting temperature will be affected. Conditions can be established to generally lower the melting temperature of dsDNA, for example, by adding urea to the gel and/or electrophoresis buffer, or conditions can be altered to lower or raise melting temperatures of specific dsDNA sequences.

Additionally, the rate at which the temperature cycles from the high temperature to the low temperature (“ramping” of temperature), can be altered to affect separation. For example, if two fragments to be separated differ in melting temperature by 1° C., a longer ramp time would improve separation. Separation will depend on the amount of time one fragment is partially denatures while the other fragment is double-stranded. By extending the ramp time of oscillations, the time during electrophoresis spent between the two melting temperatures is increased, thereby improving separation.

As a result of the invention described herein, highly sensitive and accurate methods of detecting polymorphic DNA and determining allele frequencies are now available.

### EXEMPLIFICATION

### EXAMPLE 1

### Cycling Gradient Capillary Electrophoresis: A Low-cost Tool for High-throughput Analysis of Genetic Variations

In the present work, the general principle that applying a temporal temperature gradient in CE can further be extended into applying a periodical temperature cycle is demonstrated. PCR is employed with one of the primers extended by a high-melting domain (the “GC-clamp”) to amplify a target DNA sequence surrounding the mutant or SNP marker. The PCR conditions are specific for each target sequence.

The application of periodic cycles allows a better compensation of the local temperature fluctuations inside the multi-capillary oven. Rapid gradient cycling with rates of up to several cycles per minute showed better results in comparison with slower cycling intervals. In addition, the instrument hardware does not appear to be able to follow the rapid temperature changes inside the chamber resulting in a relative constant average temperature of the optical components. Periodic application of the temperature gradient enables usage of multiple injection technology, in which different samples are serially injected between the cycles and separated under the same revolving temperature conditions. Multiple-injections allow for significant increase in sample throughput. Considering the ease of sample preparation, PCR directly followed electrophoresis without any post-PCR treatment such as desalting or removal of unincorporated primers. Evaluation of the mutant presence or SNP genotype is performed solely based on an internal standard running in a separate spectral channel. In situations where a slower migrating PCR fragment would directly overlap with a faster migrating primer peak from a subsequent injection, the spacing between the injections was adjusted. For a given mutant/SNP marker, the peak distance is very reproducible, therefore the injection spacing can be optimized accordingly. This technique represents a cost effective, simple and powerful tool for high-throughput scoring of DNA mutants and SNPs.

Chemicals

All experiments were performed using standard MegaBACE. buffers and MegaBACE. LPA long-read matrix (Amersham Biosciences, Piscataway, N.J., USA). PCR primers were obtained from MedProbe (Oslo, Norway). The primer with GC clamp were labeled with 6-carboxyfluorescein (6-FAM) on the tested samples and tetramethylrhodamine (TMR) on the internal standards. Primer used for SNP in the lymphotoxin alpha (LTA) gene (accession number 153440, locus 6p21.3, NCBI reference SNP ID: rs 909253), 5′ CGC CCG CCG CGC CCC GCG CCC GTC CCG CCG CCC CCG CCC GCT GGT GGG TTT GGT TTT GG 3′ (SEQ ID NO:1)and 5′ GAG CAG AGG GAG ACA GAG AGA G 3′ (SEQ ID NO:2). Primer used for mutation in Adenomatous polyposis coli (APC) gene (accession number 175100, locus 5q21-q22, 1p34.3-1p32.1, exon 15, codon 1450), 5′- CGG GCG GGG GCG GCG GGA CGG GCG CGG GGC GCG GCG GGC GAG CAT TTA CTG CAG CTT GCT 3′ (SEQ ID NO:3)and 5′ ACC TCC TCA AAC AGC TCA AA 3′ (SEQ ID NO:4). Primer used for SNP in the breast cancer-2 (BRCA2) gene (accession number 600185, locus 13q12.3, NCBI reference SNP ID: rs573014), 5′ CGC CCG CCG CGC CCC GCG CCC GTC CCG CCG CCC CCG CCC GAA GG TAT GTG CAT TGT TTT T 3′ (SEQ ID NO:5) and 5′ CCG CAA TAA AGC AAA TAT TAC 3′ (SEQ ID NO:6).

Capillary Electrophoresis (CE)

All CE experiments were performed on an MegaBACE 1000 96-capillary DNA analysis system (Amersham Biosciences). The instrument was equipped with an automated loading robot Caddy 1000 (Watrex Praha, Prague, Czech Republic), to allow for unattended automated operation. To reach high temperatures needed for some mutant separation, the temperature sensor was equipped with an additional resistor resulting in a positive offset of temperatures by approximately 10° C. The temperature was recorded using Fluke logging thermometer with FlukeView software (Michell Instrument, San Marcos, Calif., USA). The temperature profiles were constructed using MegaBACE configuration selector (MBCS) software (Genomac International, Prague, Czech Republic). The data was processed by SNP Profiler software (Amersham Biosciences).

PCR Reaction

Full blood from blood donors at Ulleval Hospital (Oslo, Norway) was anonymously collected and genomic DNA was extracted with QIAamp DNA Blood Mini Kit from Qiagen (Valencia, Calif., USA). All reactions were performed on a PTC-200 thermocycler (MJ Research, Waltham, Mass., USA), by mixing 50 ng of genomic DNA with 25 mM of each dNTP (Abgene, Epsom, UK), 10 Taq buffer, 1 unit of Taq polymerase (Applied Biosystems, Foster City, Calif., USA) and 5 pmol of each primer (MedProbe) in a final volume of 25 L. Same cycling conditions were applied for amplification of all fragments. The cycling program included denaturation for 60 s at 94° C., annealing for 60 s at 53° C. and elongation 60 s at 72° C., for 35 cycles. Where applicable, heteroduplexes were formed by heating the PCR products at 94° C. for 5 min, then annealing the fragments at 65° C. for 60 min followed by slow cooling to 4° C.

Optimization of Cycling Temperature Range

For each target sequence, extended by the GC clamp, the theoretical melting temperature profile was first calculated using WinMelt. simulation program (Medprobe) based on Poland's algorithm. A melt profile will show regions of theoretical high and low melting domains of a known sequence. Location of primers and GC clamps can be optimized by analyzing their effect on the overall fragment melting profile. The temperature oscillation was within ±1° C. range from the melting temperature of the low-melting domain (target sequence).

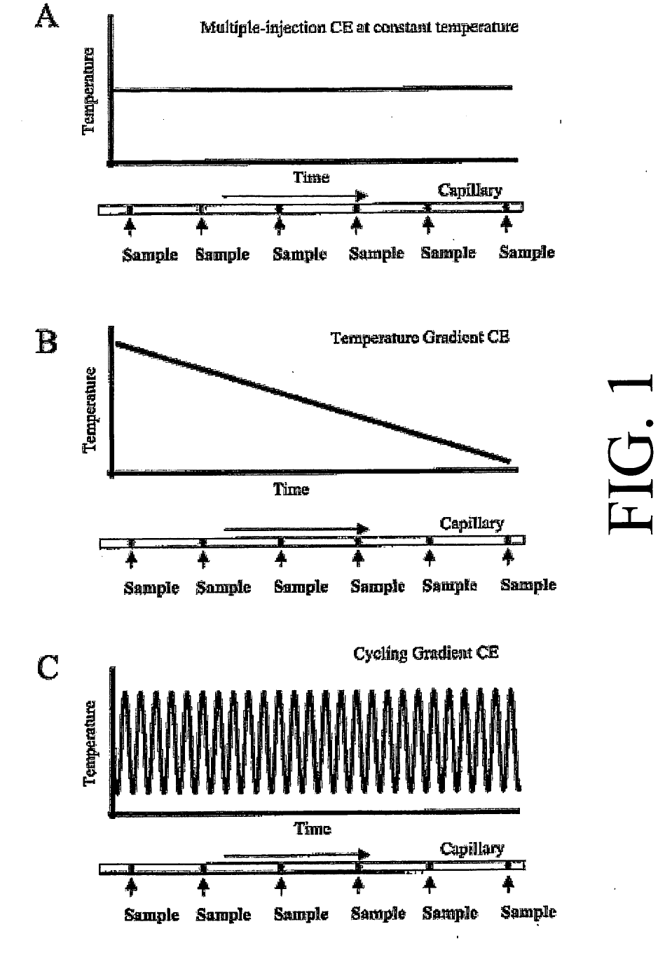

For separation of wild-type and mutant fragments based on differential melting, this condition applies. In most of these cases, the time difference between the fastest migrating peak from the mixture (usually the unincorporated primer) and the slowest migrating peak (usually the most denatured fragment) is typically around 10 min relative to the total analysis time of 30-40 min (Kristensen, A. et al., 2001, BioTechniques, 33:650-654; Zhu, L. et al., 2001, Electrophoresis, 22:3683-3687). The multiple-injection scheme could be directly applied to DNA separation, where samples would be serially injected and separated at a constant temperature (FIG. 1A).

A different situation occurs if a temperature gradient needs to be applied. In temperature gradient capillary electrophoresis (TGCE), the running temperature is gradually changed during the entire run. The fundamental assumption is that the samples have to be subjected to a proximity of optimum melting conditions over a sufficient duration of their migration in the capillary. With multiple injections, the samples eluting at the beginning would be subjected to different temperature ranges compared to the ones injected later as shown in FIG. 1B. In order to subject samples from all injections to identical melting conditions, the temperature has to be periodically changed to follow the profile of repeatedly injected samples. With this arrangement, each sample undergoes the same number of temperature gradient cycles and thus is subjected to the same melting conditions (FIG. 1C).

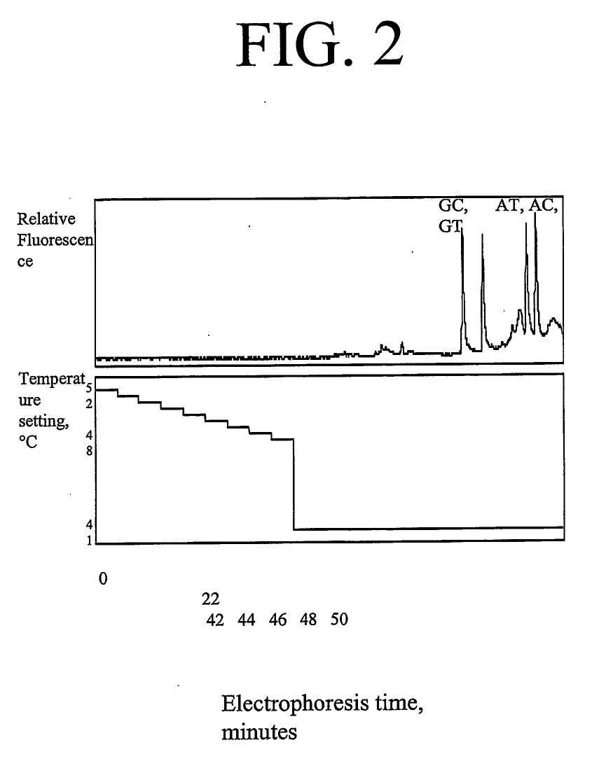

Others have subjected samples (PCR products) containing an artificial high-melting domain (“CG-clamp” or “GC-clamp”) to a temperature gradient to cover a range of optimum melting conditions (Kristensen, A. et al., 2001, BioTechniques, 33:650-654). A typical result of this single-sweep gradient experiment (TGCE) is shown in FIG. 2A. An LTA mutant was subjected to a descending temperature gradient starting at 52° C. and ending at 48° C. with a rate of 0.1° C. per min. During the gradient, the period of time at which the samples were subjected to their optimum melting conditions is given by the gradient slope and has direct impact on separation resolution (Minarik, M. et al., 2001, poster presented at ASHG Meeting, San Diego, Calif.). Described herein are methods for subsidizing this period with a series of cycles during which the separated samples are several times subjected to the melting optimum. FIG. 2B shows a separation of the same sample (LTA) subjected to a cycling temperature gradient. It can be seen that the resolution in this case is fully comparable to the single-sweep gradient experiment from FIG. 2A. It seems that the overall retardation effect due to partial denaturation in case of a cycling gradient is similar to a single-sweep gradient.

### EXAMPLE 2

### Direct Identification of All Oncogenic Mutants in KRAS Exon 1 by Cycling Temperature Capillary Electrophoresis

Over the past few decades, advances in genetics and molecular biology have revolutionized the understanding of cancer initiation and progression. Molecular progression models outlining genetic events have been developed for many solid tumors, including colon cancer. Previous reports in the literature have shown a relationship between different KRAS mutations and prognosis and response to medical treatment in colon cancer patients. Furthermore, the presence of a mutated KRAS has been correlated with different clinicopathological variables including age and gender of patients and tumor location. The mutation analysis method described herein is adapted to a 96-capillary electrophoresis instrument that allows identification of all 12 oncogenic mutations in KRAS exon 1 under denaturing conditions. To determine the optimal parameters, a series of DNA constructs generated by site-directed mutagenesis was analyzed and the migration times of all mutant peaks were measured. A classification tree was then made based on the differences in migration time between the mutants and an internal standard. A randomized series of 500 samples constructed with mutagenesis as well as 60 blind samples from sporadic colon carcinomas was analyzed to test the method. No wild-type samples were scored as mutants and all mutants were correctly identified. Post polymerase chain reaction (PCR) analysis time of 96 samples was performed within 40 min.

DNA Samples

Control mutants and the series of 500 test samples were made by PCR mutagenesis. Genomic DNA from the cell line HT 29 with wild-type sequence in KRAS exon 1 (ATCC cell repository, ATCC HB 8245) was used as template. One sense mutagenesis primer for each missense mutation in codon 12 (wild-type sequence GGT) and 13 (wild-type sequence GGC) of KRAS exon 1 was designed. The 12 mutagenesis primers were 45 base pairs long each and spanned codon 1-15 of KRAS exon 1. Each primer had a one base difference from the wild-type corresponding to the 12 different mutations. The sequence of the antisense primer was as follows: 5′ TAMRA CGC CCG CCG CGC CCC GCG CCC GTC CCG CCG CCC CCG CCC GCC TCT ATT GTT GGA TCA TAT TC 3′ (SEQ ID NO:7; products used for internal standard) or 5′ 6-FAM CGC CCG CCG CGC CCC GCG CCC GTC CCG CCG CCC CCG CCC GCC TCT ATT GTT GGA TCA TAT TC 3′ (SEQ ID NO:7; control samples and series of 500 test samples). All mutagenesis PCR products were sequenced on a MegaBACE 1000 (Amersham Pharmacia Biotech, Oslo, Norway) to verify correct sequence, with conditions recommended by the manufacturer and 5′ CGC CCG CCG CGC CCC GCG CC 3′ (SEQ ID NO:8) as sequence primer. DNA from 60 sporadic colon cancers was extracted with a standard DNA extraction kit (Qiagen, Valencia, Calif., USA).

Thermodynamic

KRAS exon 1 with attached GC-clamp was analyzed with computer program MacMelt (MedProbe, Oslo, Norway). The program calculates the thermodynamics of the transition of double-stranded to single-stranded DNA (Fixman, M. and Freire, J., 1977, Biopolymers, 16:2693-704)

PCR

All PCR reactions were performed on a PTC-200 thermocycler (MJ Research, Waltham, Mass., USA). The cycling parameters were 35 cycles of denaturation for 1 min at 94° C., annealing at 53° C. for 1 min and elongation at 72° C. for 1 min, followed by heteroduplex formation by boiling and re-annealing at 65° C. for 30 min. PCR amplification was performed by mixing 50 ng genomic DNA or 0.01 μL of PCR product with 25 μM of each dNTP (Perkin Elmer, Oslo, Norway), 10 Taq buffer, 1 unit cloned Taq (Stratagene, La Jolla, Calif., USA), and 5 pmol of each primer (MedProbe) in a final volume of 25 μL. PCR primers for amplification of KRAS exon 1 in the tumor samples were as follows: 5′ ATG ACT GAA TAT AAA CTT GTG 3′ (SEQ ID NO:9) and 5′ 6-FAM CGC CCG CCG CGC CCC GCG CCC GTC CCG CCG CCC CCG CCC GCC TCT ATT GTT GGA TCA TAT TC 3′ (SEQ ID NO:7).

Standard

An internal standard with five peaks was created by mixing TAMRA-labeled PCR products from three homozygous mutant samples (a codon 12 GGT to TGT mutant, a codon 12 GGT to CGT mutant and a codon 13 GGC to AGC mutant) with the unlabeled wild-type PCR product. The product mixture was thereafter boiled and re-annealed for heteroduplex formation. Since only the mutant samples were labeled prior to mixing and two heteroduplexes co-migrated, five peaks were visible when the standard was analyzed with CTCE. The standard was added to all wells in the plates prior to CTCE analysis of test samples.

CTCE

Samples were analyzed by CTCE in a standard 96-capillary DNA sequencing instrument, the MegaBACE. 1000 DNA Analysis System (Amersham Biosciences, Uppsala, Sweden). The distance from the anode to the detector was 40 cm. Regular 3% linear polyacrylamide (LPA) with urea (7 M) was replaced prior to every run by applying high-pressure nitrogen. PCR products were diluted 1:25 in water prior to electrokinetic injection, accomplished by applying 10 kV for 12 s. The electrophoresis condition was a constant field of 150 V/cm. The partial melting of DNA fragments was achieved by a combination of urea in the gel and temperature surrounding the capillaries. The separating temperature used was a gradient going from 51° C. down to 49° C. in 1° C. increments. Each temperature was held for 30 s. The gradient was cycled 15 times before the product reached the detector. The gradient was followed by constant temperature at 44° C. until all peaks had eluted. All samples were analyzed in 96-well plates. In the presented case, laser-induced fluorescence detection was used with excitation at 488 nm (blue laser) and detection of emission from the 6-FAM-labeled PCR primer at 520 and excitation at 532 nm (green laser) for the TAMRA-labeled PCR primer with detection of emission at 580 nm. By using these PCR primer labels no spectral overlap was observed between the channels. Migration times of all peaks in the electropherograms were scored by computer program Genetic Profiler and the relative migration times between peaks were calculated.

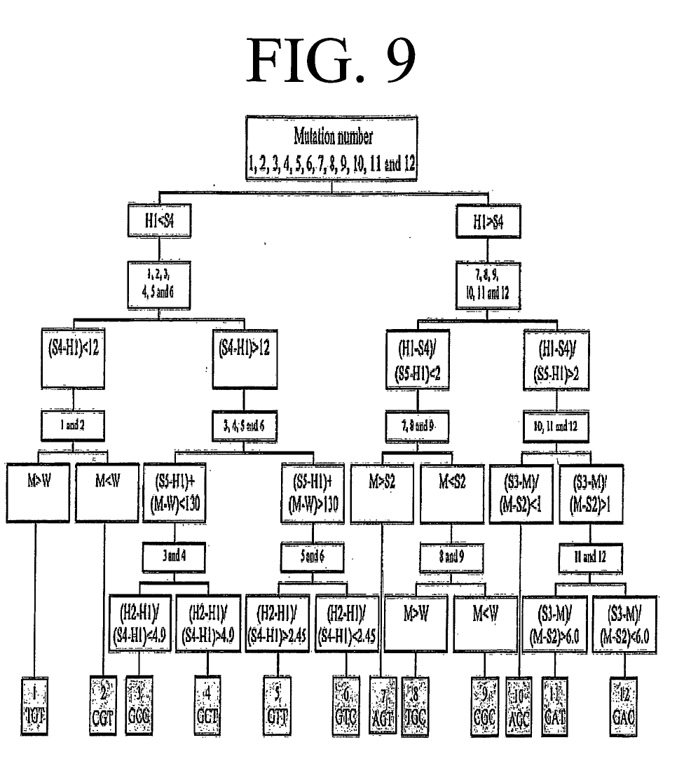

Classification Tree A plate with 96 control samples (12 mutants times 8) was used to make a classification tree. Each well in the plate contained the TAMRA-labeled standard and one 6-FAM-labeled control sample. The plate was analyzed by CTCE under various denaturing conditions where temperature and number of cycles were adjusted until optimal reproducibility was obtained. At denaturing conditions with high reproducibility, the migration times of wild-type and mutant peaks, as well as migration times of the five standard peaks were measured in each electropherogram by Genetic Profiler computer software (Amersham Biosciences). Migration times from eight runs for each mutant were used as references. The migration time data was used to generate a classification tree by extrapolating from the compiled empirical data (FIG. 9). Identification of the unknown mutants was achieved by comparison of migration differences between sample and standard peaks and related to the parameters in the classification tree. It is crucial that the classification tree is followed from top to bottom for correct identification of the mutants. The letters in the classification tree boxes indicate different peaks as noted in FIG. 9. For example, a GGT to TGT mutation in codon 12 of KRAS has the following characteristics: (i) the first heteroduplex (H1) has a shorter elution time than standard peak 4 (S4), (ii) the migration time of S4 minus migration time of H1 is less than 12, and (iii) the migration time for the mutant peak (M) is longer than the migration time for the wild-type peak (W). The flow chart was used to identify the mutant sequence in 500 blindly scored samples and 60 sporadic colon carcinomas with known mutation status.

Results and Discussion

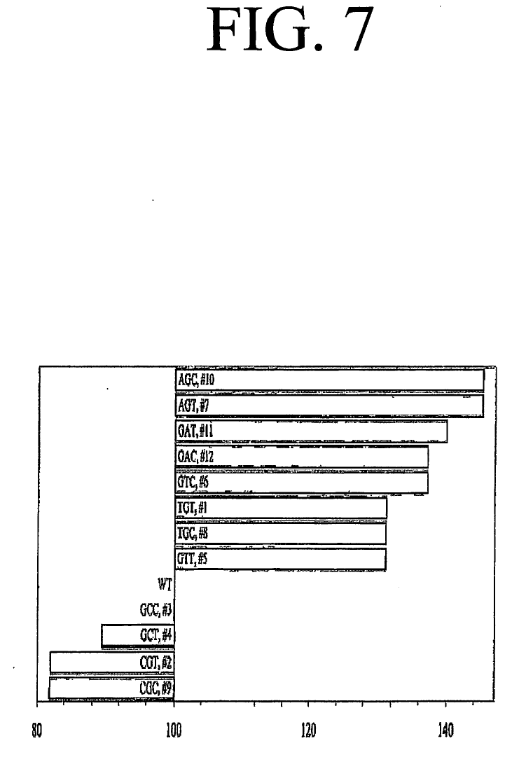

A theoretical melting profile analysis of the 151 base pair target fragment, including exon 1 of KRAS and the PCR-attached GC-clamp was first performed. All oncogenic mutations in KRAS exon 1 as well as wild-type were analyzed. The relative differences in thermodynamic properties between the mutations and wild-type fragments are illustrated in FIG. 7. Wild-type KRAS exon 1 with the sequence GGT and GGC in codons 12 and 13, respectively, is coded as 100% in FIG. 7. All but one G to C mutation proved to be more stabile than the wild-type sequence, and thus separated out before wild-type peaks in electropherograms. A GGC to GCC mutation, which was only slightly more stable than wild-type, co-migrated with the wild-type. All other mutations were less stable than wild-type and eluted after the wild-type peak. Some of the mutations (e.g., AGC and AGT, and TGT, TGC and GTT) had very similar melting profiles.

Based on the theoretical thermodynamics of the different fragments a standard was designed. Three mutations with similar properties to other mutants were used in the internal standard. The inclusion of these mutations in the standard allowed for identification of mutants with almost equivalent thermodynamics. The second step required for the CTCE setup was the determination of the optimum denaturing conditions for the separation of homoduplexes and heteroduplexes by performing several runs with different numbers of temperature cycles and different temperature intervals. The goal was to find denaturing conditions that allowed separation corresponding to the theoretical thermodynamics of all mutants. Highly reproducible separation of mutant homoduplex peaks was achieved by a gradient ranging from 51° C. down to 49° C. in steps of 1° C. Each temperature was held for 30 s. The gradient was cycled 15 times before the product reached the detector. Due to local temperature differences in the capillary chamber, few capillaries will be exposed to the optimal separating temperature if the temperature was held constant during electrophoresis. By cycling the temperature around the theoretical optimal separation temperature, all capillaries will be exposed to the “optimal” temperature, during the temperature gradient created in the capillary chamber during electrophoresis. The more times the gradient is cycled the longer each capillary will be under optimal separation conditions. If the gradient is cycled to too rapidly the heat capacity of the chamber will not have time to adjust, thus making suboptimal denaturing conditions. By cycling the gradient few times, or keeping the temperature at the same interval in the cycle for a longer time, suboptimal denaturing conditions are observed in some capillaries. Methods for CDCE of KRAS exon 1 applied to a MegaBace 1000 had been reported (Bjørheim, J. et al., 2002, Anal. Biochem., 304:200-205). Although the methods proved sensitive and specific in mutation detection it was not possible to directly identify the mutations by this method. Consequently, mutation verification had to be performed by mixing of samples with controls prior to re-analysis.

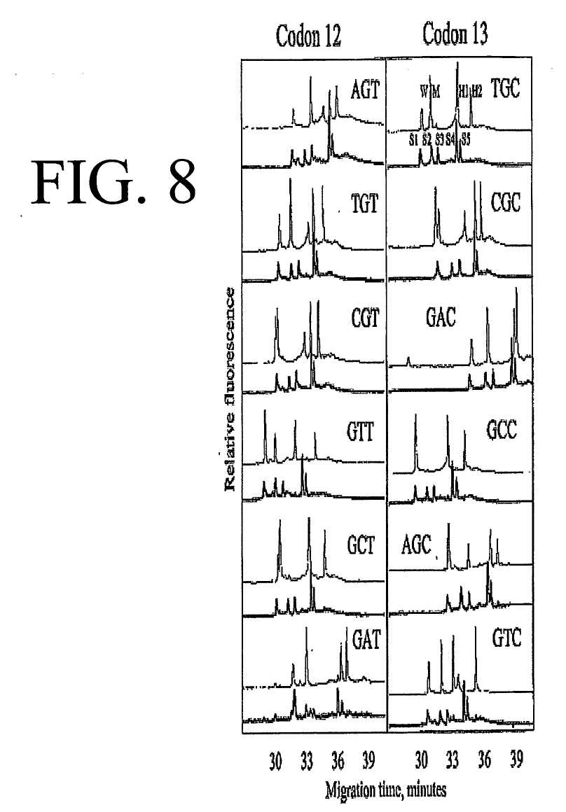

With the selected denaturing conditions eight 96-well plates with standard and different mutations and wild-type samples were analyzed by CTCE. Separation between the wild-type homoduplex and the heterozygous peaks was achieved in all mutated samples. Separation between the wild-type homoduplex and mutant homoduplex was found in 11 out of 12 mutants. The mutant homozygous peak of GGC to GCC transversion in codon 13 co-migrated with wild-type at these denaturing conditions. These observations corresponded with the theoretical thermodynamics of the different fragments. Electropherograms of all mutant samples and the standard are shown in FIG. 8.

No spectral overlap was observed between the channels. Individual mutations proved to have distinct migration times when compared to the peaks of the internal standard and other mutations. Migration times that were able to separate different mutations were selected and a classification tree was made (FIG. 9). For example, a TGT mutation was characterized first by a shorter migration time of H1 than S4, and migration time of S4minus migration time of H1 was less than 12. Finally, the migration time of the mutant homoduplex (M was longer than for the wild-type peak (WT). No other mutations had these characteristics.

To test whether blind samples could be scored and identified based on peak migration times and the classification tree, 500 samples with wild-type and mutant sequence in codon 12 and 13 of KRAS were scored. The samples were PCR amplified by another investigator and analyzed without any knowledge of the samples. All 500 samples analyzed were scored correctly. No wild-type samples were scored as mutants and no mutant sample was placed in the wrong group. To further investigate the adaptability of the method to clinical use, 60 sporadic colon carcinomas were analyzed. A total of 22 samples were found mutated in KRAS exon 1 and all mutants were scored correctly when compared to previous CDCE analysis of the samples. Among the 560 samples used to evaluate the method, 100% sensitivity and specificity were demonstrated.

Other reports on mutation analysis of KRAS with automated CDCE have demonstrated a detection limit as low as 0.1% for heterozygous peaks and down to 1% for homozygous mutant peaks. Similar detection limits were found in this setup (data not shown). Since the decision making tree is dependent on mutant homoduplex peaks for mutation identification, mutants with mutated allele fraction below 1% are not identified in this system. To identify a mutant below the 1-% level, the mutant was mixed with a control mutant, denatured and thereafter re-annealed prior to CTCE analysis (Guldberg, P. and Guttler, F., 1993, Nucleic Acids Res., 21:2261-2262) as reported for other melting gel techniques. Evaluation of the detection limit was performed by the formula as described by Ekstrøm et al. (2000, BioTechniques, 29:582-589).

Many methods have been employed to detect mutations. In one approach, PCR is part of the detection system. Two widely used methods, allele-specific amplification and mutant-enriched PCR, can detect one mutant allele against a background of 10 000 wild-type alleles (Nollau, P. and Wagener, C., 1997, Clin. Chem., 43:1114-1128; Andersen, S. et al., 1999, Gut, 45:686-692.). Although the sensitivity of such methods is high, they are not amenable to automation and high throughput. In a second type of approach, mutations are analyzed after the target sequence is amplified by PCR and detected using techniques such as ligation. The main disadvantage of ligation is that three pairs of oligonucleotides are needed to identify a bi-allelic marker (Lehman, T. et al., 1996, Anal. Biochem., 239:153-159.). Interestingly, PNA-directed PCR in combination with MALDITOF reports high sensitivity in KRAS detection, and the possibility for automation. However, several steps are needed to identify mutant samples and identification of a possible mutant in codon 12 must be evaluated prior to codon 13-mutation status analysis (Sun, X. et al., 2002, Nat. Biotechnol., 20:186-189).

The MegaBACE 1000 with 96 capillaries in parallel analyzed 96 samples under denaturing conditions within 40 min. A very limited number of steps were needed in this setup. The samples were PCR-amplified in 96-well plates and diluted 1:25 in water. Thereafter, without any modifications, the plates were ready for CTCE analysis. All codon 12 and 13 mutations were directly scored and identified, thus eliminating the need for further evaluation.

CTCE methods of the present invention offer two major advantages over previously reported techniques adapted to KRAS mutation analysis. The first advantage allows for direct identification of all oncogenic KRAS mutations in exon 1 without the need for sequencing. Secondly, the short post-PCR analysis time makes this technique highly desirable for fully automated mutant screening.

### EXAMPLE 3

### Population Screening of Single-Nucleotide Polymorphisms Exemplified by Analysis of 8000 Alleles

Described herein is a method in which the population frequency of single-nucleotide polymorphisms (SNPs) can be efficiently detected and their allele frequencies accurately measured. Selected SNPs in TNFβ, IL-4, and CTLA-4 were used to demonstrate the method. Blood from 4000 individuals was pooled, DNA was extracted, and target sequences were PCR-amplified and analyzed by denaturant capillary electrophoresis. Alleles were separated into peaks based on melting properties of the double DNA helix. Frequencies of the different alleles were determined by calculating the area under the peaks. Allele frequencies and Hardy-Weinberg equilibrium estimated from the pooled data were verified by analyzing 7.5% of the samples randomly selected from the blood donor series. The method herein is suitable for single-samples and/or pooled-samples analysis of SNPs, in which sample treatment is kept to a minimum. The potential throughput of the method is beyond obtainable numbers of samples.

SNPs are considered to be useful polymorphic markers for genetic studies of pharmacogenetics and polygenic traits (McCarthy, J. and Hilfiker R., 2000, Nat. Biotechnol., 18:505-508; Roses, A., 2000, Nature, 405:857-865). A worldwide effort to collect SNPs has achieved an accumulation of millions in public databases. However, most of these SNPs have been identified by examination of a limited number of individuals, and information on their allele frequencies is lacking or tentative. Furthermore, studies have shown that allele frequencies vary widely between different ethnic populations. Thus, validation of SNPs and estimation of their allele frequencies, especially for each ethnic group, are required before these markers can be used for genetic studies (Risch, N., 2000, Nature, 405:847-856).

Pooling of samples is an obvious approach for frequency determination of SNP in large populations because it drastically reduces the cost of the analysis and labor time compared with genotyping individuals and counting alleles. The quantification method in the pooled analysis must accurately reproduce the genotype frequencies found when analysis of single samples is performed.

Described herein is a method for allele frequency estimation of SNPs, based on, for example, in part on denaturant capillary electrophoresis (DCE). DCE is based on the melting gel theory described by Fischer and Lerman (Fischer, S. and Lerman L., 1983, Proc. Natl. Acad. Sci. USA, 80:1579-1583). Double-stranded DNA fragments melt characteristically based on the nucleotide sequence and length of the fragment when exposed to denaturants such as temperature, urea, or formamide. Fragments with only minor differences to the wild-type, such as a base substitution, result in different melting characteristics. Due to the high sensitivity and simplicity described for melting gel techniques applied on capillary electrophoresis (Khrapko, K. et al., 1994, Nucleic Acids Res., 22:364-369) this method was found suitable for population frequency analysis of SNP.

Whole blood from 4000 donors was prepared in 4 pools, with 1000 samples in each pool, prior to DNA extraction. The pooled samples and 7.5% of the individual DNA samples were subjected to PCR subsequently followed by DCE analysis. Allele frequencies were determined in the pooled samples by measuring the area under the peaks in the electropherograms. The individual samples were genotyped by comparing the peak pattern with a heterozygous standard run simultaneously with a different fluorochrome. The throughput of this SNP approach is limited by the number of samples obtainable. In simple terms, 96 pools with 1000 samples in each, totaling 96,000 samples, can be analyzed in 35 minutes. For single-samples analysis, the theoretical throughput is about 46,000 samples in 24 hours in a fully automated system. This simple 2-step protocol, PCR and electrophoresis on standard capillary DNA sequencing equipment, could make a significant contribution to SNP analysis.

Pooled Samples

Blood samples from 4000 blood donors were collected from August to October 2000 within a 10-week interval to exclude parallels from the same individual. The sampling was anonymous; no record exists that can link the blood sample to the donors. White blood cells were not counted due to the statistical observation described in the central limit theorem.

$$Z = \frac{\overline{X} - μ}{σ / \text{?}}$$

$$\text{?} \text{indicates text missing or illegible when filed}$$

is approximately N(0,1).

The theorem states that in a random sampling of a population with mean μ and standard deviation of σ, the distribution of average X when n is large is approximately normal, with mean μ and standard deviation σ/square root of n (Bhattacharyya, G. and Johnson. R., “The normal distribution and random samples.” In Bradley R A, Kendall D G, Hunter J S, Watson G S (eds): Statistical Concepts and Methods. New York: John Wiley, 1977:187-232). Consequently, the average number of white blood cells for each allele will approach the average.

Two pooling steps made 4 pools with blood from 1000 donors in each. First, 50 μL blood from 100 donors was pipetted into a tube and mixed vigorously. Subsequently, 1 mL blood from 10 pools made up of 100 donors was mixed, giving 4 pools with 1000 samples in each. DNA was extracted from the pools by use of the Qiagen Max Blood extraction kit (Qiagen Inc., Valencia, Calif.). All single samples subjected to DNA extraction from the blood donor samples were extracted with a GenoM™-48 (Genovision, Oslo, Norway), and aliquots were transferred into 96-well microtiter plates.

PCR Reactions

All reactions were performed on a PTC-200 thermal cycler (MJ Research,Waltham, Mass.) by mixing 50 ng of pooled- or individual-sample DNA with 25 mM of each dNTP (Perkin Elmer, Oslo, Norway), 10× Taq buffer, 1 unit of Taq polymerase (Perkin Elmer), and 5 pmol of each primer (MedProbe, Oslo, Norway) in a final volume of 25 μL. The same cycling parameters were used for the 3 fragments—denaturation 60 seconds at 94° C., annealing 60 seconds at 53° C., elongation 60 seconds at 72° C.—for 35 cycles.

The PCR products were denatured 5 minutes at 94° C. and incubated 30 minutes at 65° C. for heteroduplex formation.

Primers
TNFβ, Genbank accession number M55913, variation
in base pair 329 A/G:

(SEQ ID NO:1)

5′ CGC CCG CCG CGC CCC GCG CCC GTC CCG CCG CCC CCG
CCC GCT GGT GGG TTT GGT TTT GG 3′;

(SEQ ID NO:2)

5′ GAG CAG AGG GAG ACA GAG AGA G 3′.
IL-4, Genbank accession number AF395008, variation
in base pair 12559 T/C:

(SEQ ID NO:10)

5′ CGC CCG CCG CGC CCC GCG CCC GTC CCG CCG CCC CCG
CCC GCT ATC TTT GTC AGC ATT GCA T 3′;
5′ ATG CTA GCA GGA AGA ACA GA 3′
CTLA-4, Genbank accession number AF414120, varia-
tion in base pair 204 A/G:

(SEQ ID NO:11)

5′ CGC CCG CCG CGC CCC GCG CCC GTC CCG CCG CCC CCG CCC
GCT TCC TGA AGA CCT GAA CAC 3′;

(SEQ ID NO:12)

5′ CAG GGA TGA AGA GAA GAA AA 3′.

The primers with GC-clamp were labeled with 6-carboxyfluorescein or Tamra fluorochromes.

Denaturant Capillary Electrophoresis

The pooled samples were analyzed by the ABI 310 Genetic Analyzer (Applied Biosystems, Foster City, Calif.) under constant denaturing conditions. Temperature of the capillary was set to 52° C., 47° C., and 56° C. (TNFβ, IL-4, CTLA-4, respectively).

Single samples were analyzed by temperature gradient capillary electrophoresis (TGCE) in a 96-capillary system. The instrumentation was a standard multi-capillary DNA sequencing instrument MegaBACE™ 1000 DNA Analysis System (Amersham Pharmacia Biotech, Oslo, Norway). The instrument is a high-throughput, fluorescence-based DNA system using capillary electrophoresis with 96 capillaries in parallel. The distance from the anode to the detector is 40 cm. The MegaBACE™ platform uses cofocal laser scanning focused on each capillary. Regular 3% linear polyacrylamide (LPA) with urea was replaced prior to every run by applying high-pressure nitrogen. PCR products were diluted 1:25 in water prior to electrokinetic injection, accomplished by applying 10 kV for 12 s. The electrophoretic condition was a constant field of 150 V/cm. The partial melting of DNA fragments was achieved by a combination of urea in the matrix and temperature surrounding the capillaries.

TNFβ was separated with a gradient going from 54° C. down to 52° C. in steps of 0.5° C., and each temperature was held for 210 seconds. The gradient was followed by constant temperature at 50° C. until the end of the run.

The gradient used to separate the C and T alleles in IL-4 was 51° C., 50° C., and 49° C., and each temperature was held for 2 minutes. The gradient was cycled 4 times, followed by constant temperature at 48° C. until the end of the run.

CTLA-4 alleles were separated by a cycling gradient of 56° C., 55° C., and 54° C. (1 min each temperature) 7 times, followed by constant temperature at 50° C.

The areas under the peaks were measured with the Acknowledge computer program (Biopac Systems, Inc., Santa Barbara, Calif.). To detect potential PCR bias between the 2 alleles, peak areas in individual heterozygous samples were measured.

Selected samples were sequenced to verify the genotype, using the first 20 bases of the GC-clamp primer. The sequencing reaction and analysis were set up as described by the manufacturer (Amersham Pharmacia Biotech, Oslo, Norway).

Control of PCR Bias

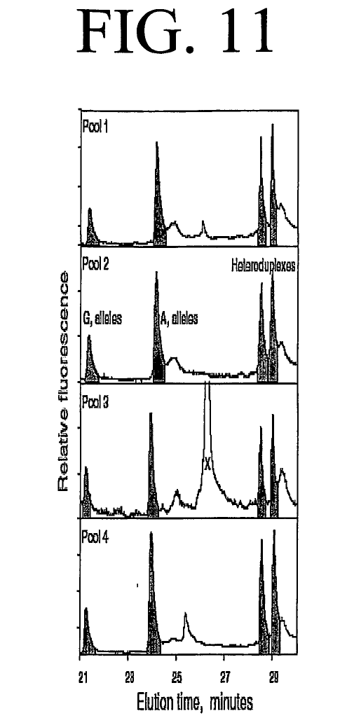

Analysis of PCR-amplified DNA introduces the possibility of preferential amplification of different alleles. PCR bias was analyzed by measuring the peak areas in heterozygous samples. No preferential amplification of alleles was observed when areas under the peaks were measured in heterozygous individual samples. FIG. 11 represents 5 individual heterozygous samples in TNFβ analyzed by TGCE in different runs; the average ratio between the peak area was 0.996 (one SD=0.028). The average ratio between the 2 alleles in IL-4 and CTLA-4 was 0.994 (one SD=0.06) and 0.993 (one SD=0.06), respectively.

Pooled Samples

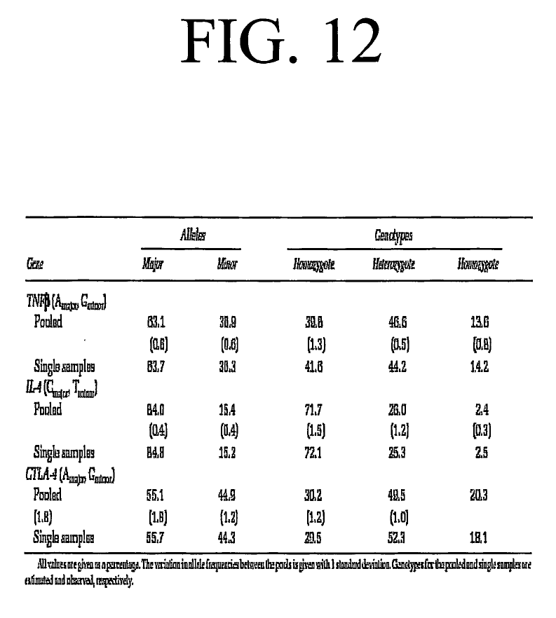

In the four pools, each allele frequency was determined by measuring the peak areas representing each allele. FIG. 2 shows the electropherograms of the four pools analyzed for the selected TNFβ polymorphism. The results from the pooled blood samples are summarized in FIG. 12. For the TNFβ polymorphism, the frequency was 63.1% and 36.9% for A alleles and G alleles, respectively. The mean allele frequency for IL-4 was 84.6% for C alleles and 15.4% for T alleles. For CTLA-4, the frequencies found were 55.1% for A alleles and 44.9% for G alleles.

Individual Samples

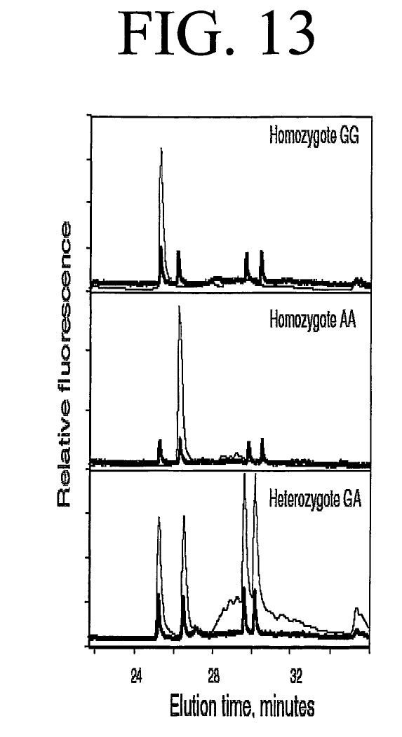

Genotypes of 300 individual samples were determined by coelution with an internal heterozygous carboxytetramethylrhodamine (Tamra)-labeled standard. In FIG. 13, the genotypes in the TNFβ SNP are depicted with GG at the top, AA in the middle, and GA at bottom. The peaks presented by the bold line are a Tamra-labeled standard run simultaneously in each capillary. There was no need for spectral overlap corrections due to the use of 488-nm and 532-nm laser excitation of 6-caroxyfluorescein and Tamra, respectively. In FIG. 12, the allele frequencies for the 3 SNP measured in the pooled samples are compared with the single-samples analysis. For TNFβ polymorphism, frequency was 63.7% and 36.3% for A alleles and G alleles, respectively. The mean allele frequency for IL-4 was 84.8% for C alleles and 15.2% for T alleles. For CTLA-4, the frequencies found were 55.7% for A alleles and 44.3% for G alleles. The Hardy-Weinberg equilibrium was calculated from the allele frequencies obtained from the pooled data. The estimated genotype distributions were found equal to the observed genotype frequencies from the single-samples data.

Sample Throughput

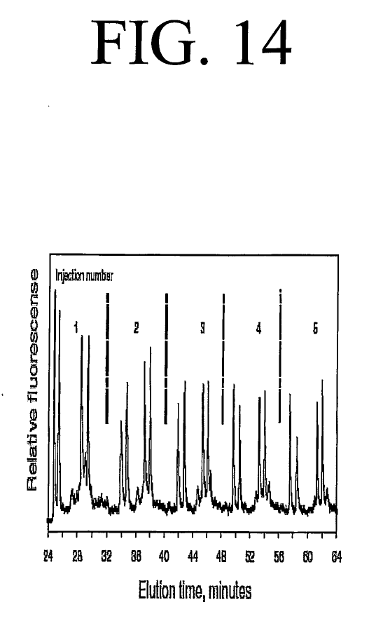

Multiple injection of samples during electrophoresis increased the throughput of the method. New samples were injected in each capillary with a 8.5 minute interval. FIG. 14 displays the electropherogram after injections of 5 samples.

To test whether the method was amenable on a 384-capillary instrument, the samples were re-analyzed on a MegaBACE™ 4000. The denaturing conditions were controlled with the same gradient used on the 96-capillary instrument. No apparent differences were observed between the instruments (data not shown). This opens a theoretical throughput of 7680 samples per hour (5 injections×4 channels×384 wells).

Discussion

SNP discovery has resulted in large databases containing information on DNA variation in millions of sequence positions of the human genome. Most SNPs reported are detected by analyzing a limited number of individuals, and the distribution frequency of an SNP is often based on small populations. Furthermore, it is likely that a broad part of the SNPs reported has no or limited impact within medicine and biology. SNPs that turn out to be correlated to aberrant observations are described in several reports and most likely will be added to the list of important SNPs in years to come. Medical intervention could be dependent on SNP knowledge so that severe side effects and inefficient treatment can be avoided. Further knowledge of SNP allele frequency distribution in different populations will be needed for optimal treatment of the individual patients and for certain subgroups of patients.

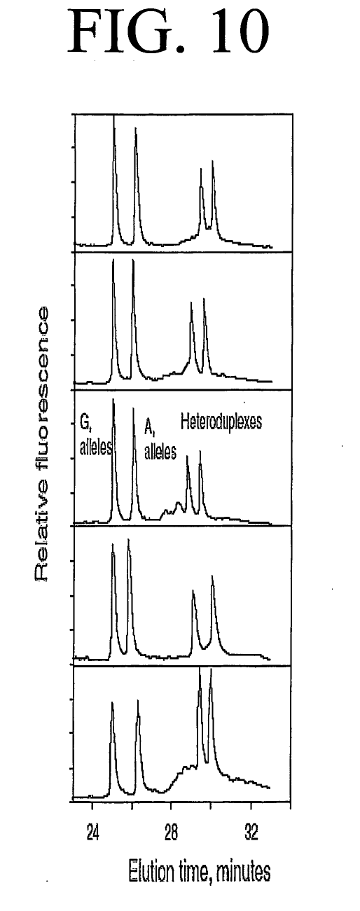

Described herein is a method for population frequency analysis of SNP. Pooled samples were subjected to denaturant capillary electrophoresis in order to measure allele frequencies of defined SNPs. Single-samples analysis was performed to confirm the allele frequencies found in the pooled samples. By pooling large groups of samples, several technical problems have to be addressed. Preferential amplification of one of the alleles during PCR would give a false relative ratio between the two alleles. No difference in amplification of the different alleles within the target sequences of heterozygous single samples could be observed (FIG. 10). Ratio of peak areas was close to 1, and thus no preferential amplification took place. Consequently, peak areas measured in the pooled-samples electropherograms represent allele copies entered in the PCR reaction (FIG. 11).

Second, when quantifying alleles by laser-induced fluorescence and electrophoresis, the peaks must be within the dynamic range of the detector, and the fluorescence signal should reflect the total copy number. Fragments that differ by a base substitution or mismatch (heteroduplexes) will pass the detector with the same velocity when analyzed below optimal separation conditions (Ekstrøm, P. et al., 2000, BioTechniques, 29:582-589). Thus, the fluorescence signal will represent the number of DNA copies.

Reports on the analysis of pooled DNA have been restricted to analysis of pools made up of equal molar of DNA. By pooling 1000 samples of whole blood or DNA, no prior knowledge of white blood cell count or DNA concentration of the individual samples is needed. Given that variance of white blood cell count or DNA concentrations is finite, the average number of each allele will approach the mean as given by the central limit theorem.

The allele frequencies estimated from the pooled data are given in FIG. 12. The standard deviation (given in parentheses) reflects the reproducibility of the method and the difference between the estimate from each pool. Comparing allele or genotype data estimated in the pools with frequencies obtained through single-samples analysis validates this pooling strategy. By using a standard DNA sequencing instrument, information on SNP population frequencies can be obtained with minimum effort. Either 96 different ethnic groups or disease cohorts of interest made up of 1000 samples each can be analyzed within 35 minutes. Furthermore, this approach was performed on standard sequencing equipment accessible for most of the research community. The samples processing is simple, involving only the almost mandatory PCR step followed by denaturant capillary electrophoresis. There is no need for post-PCR handling such as purification, hybridization, or restriction enzyme cutting, so hands-on time is taken to a minimum. As a consequence of the simple protocol, there is no need for expensive consumables as reported for other techniques. As long as the target sequence consists of a high and low melting domain (naturally or constructed) and can be amplified by PCR, virtually any DNA sequence can be analyzed for SNPs with this method. The sensitivity of the method has been described previously and opens the possibility of detecting SNPs of all frequencies (an SNP is defined to be present in more than 1% of the population) in pooled samples.

Single-samples analysis was performed by DCE in a 96-capillary sequencing instrument. Initially, constant denaturing conditions were attempted, but large capillary variability with respect to allele separation was observed (data not shown). Thus, appropriate cycling gradients were used to compensate for temperature differences within the analysis camber and between the capillaries. Alleles were determined by coelution with a heterozygous standard (FIG. 13). The clear baseline separations of the two alleles in FIG. 13 enable automated peak recognition and allele scoring. Single-samples throughput can further be increased by repeated injection of samples in the same capillary (FIG. 14). Theoretical foundations for this approach are described elsewhere (manuscript in preparation). In the laboratory, only two main steps have to be addressed: a conventional PCR amplification of the target sequence and DCE analysis. Both steps are ready to be automated by liquid-handling robotics and automation of capillary DNA sequencing instruments. The limited number of steps needed for analysis reduces the analysis time, and the risk of introducing false results is lowered.

No new method introduced in SNP analysis can omit the question about the potential cost in the case of high-throughput analysis. The pooling strategy reduces the cost 1000-fold as compared to single-samples analysis. Furthermore, the method presented herein can reduce the cost of single-samples PCR reaction 100-fold. The DNA sequencing instrument needs to handle 5- to 10-μL volumes; when taking into account the 25-fold dilution factor in H₂O of the PCR product, it would be sufficient for analysis purposes only to use the 200-nL PCR product. Consequently, PCR cost can be reduced significantly with the use of nano PCR equipment. Furthermore, the electrophoresis cost can be reduced 20-fold by using the instrument to its full capacity by multiple injections and using all four channels. Clearly, this simple and inexpensive protocol may be advantageous in analyzing large cohorts.

### EXAMPLE 4

### DNA Variants in the ATM Gene are not Associated with Sporadic Rectal Cancer in a Norwegian Population-based Study

A large number of DNA single-nucleotide polymorphisms (SNPs) have been discovered following the Human Genome Project. Several projects have been launched to find associations between SNPs and various disease cohorts. Described herein is an examination of the possible association between the reported SNPs and sporadic rectal cancer. It has been proposed that SNPs in the ataxi-telangiectasia mutated (ATM) gene modulate the penetrance of some cancers. The investigated target sequence harbors three polymorphisms (IVS38-8 T/C in intron 38, 5557 G/A and 5558 A/T in exon 39), resulting in eight possible microhaplotypes at the DNA level. Furthermore, the two exonic SNPs are sited next to each other, allowing four possible amino acids in the same codon.

Using CTCE as described herein, SNPs and microhaplotypes based on theoretical thermodynamics and migration of variant fragments are employed. Fluorophore-labeled PCR products were analyzed without any post-PCR steps on a standard 96 capillary-sequencing instrument under denaturing conditions. More than 7000 alleles were microhaplotyped based on peak migration patterns of individual samples and sequencing results. The ATM polymorphisms and microhaplotypes examined did not significantly differ between sporadic rectal cancer and normal population.

The SNP analysis method used in this study employs the following: (a) PCR amplification of the target sequence followed by (b) allelic discrimination by DNA melting equilibrium. In this study, three polymorphisms reported in intron 38 and exon 39 of the ATM gene within a 100-bp fragment for SNPs and their microhaplotypes are analyzed. A total of 3,526 samples from a normal population and 151 samples from sporadic rectal cancer patients were examined to test the method and establish microhaplotype frequencies in the respective populations.

Study Population

Tissue samples from 151 patients (90 men, 61 women) with sporadic rectal cancer were collected at the Norwegian Radium Hospital between 2000 and 2002. Medical records verified a diagnosis of rectal cancer (n=151). Patients' ages ranged from 26 to 87 years with a mean age of 65. All samples were collected after informed consent. None of the patients had a family history of colorectal cancer. The control population consisted of 3,525 anonymous blood donors serving the blood bank at Ulleval hospital, Oslo, Norway. The age distribution and gender are therefore unknown. Whole blood samples were collected within a narrow time frame (10 weeks) to exclude sampling of duplicates.

DNA Extraction

Tissue was preserved in RNA-later (Ambicon, Austin, Tex., USA) until DNA extraction was performed. DNA was extracted from tumor tissue with QIAamp DNA Kit (Qiagen, Valencia, Calif., USA). DNA was extracted from blood by used of GenoM-48 (GenoVision, Oslo, Norway) magnetic bead DNA extractor. Aliquots (100 μL) of extracted DNA were dispensed into 96-wells plates (Abgene, Epsom, United Kingdom) prior to PCR.

Polymerase Chain Reaction

Primers for the target sequence in ATM gene (GenBank accession number U82828.1, bp position 92313.92461) were designed by the primer design program Primer 3 (Whitehead Institute, Cambridge, Mass.), and the thermodynamics of the fragments was analyzed by the WinMelt computer program (Lerman, L. and Silverstein, K., 1987, Methods Enzymol., 155:482-501). A 60-mer carboxyfluorescein (6-FAM)-labeled primer with GC-clamp (5′ 6-FAM-GCG GGC GGC GCG GGG CGC GGG CAG GGC GGC GGG GGC GGG C-TC AAA CTA TTG GGT GGA TTT G 3′; SEQ ID NO:13) and a 20-mer primer (5′ TCC CTG AAC ATG TGT AGA AAG C 3′; SEQ ID NO:14) were used for PCR amplification. A TAMRA-labeled primer (5′ TAMRA-GCG GGC GGC GCG GGG CGC GG 3′; SEQ ID NO: 15) was used in place of the 5′ 6-FAM-labeled primer, to create an internal standard by re-amplifying a heterozygous samples of the two most frequent microhaplotypes (IVS38-8T-5557G-5558A, IVS38-8T-5557A-5558A). PCR reaction mixtures consisted of 50 ng genomic DNA, 0.06 U/μL Taq polymerase, 1×Buffer (ABgene) containing 2.5 MM MgCl₂, and 0.4 mM dNTP mix (ABgene). Each of the primers was added to a final concentration of 0.24 mM. The total volume was adjusted to 25 μL with sterile ion-exchanged MilliQ water (Millipore, Oslo, Norway). Amplification was performed in an air-thermocycler Peltier Thermal Cycler PTC 200 (MJ Research, Waltham, Mass., USA), using the following cycling conditions: 5 minutes at 94° C., followed by 35 cycles of 30 seconds at 94° C., 30 seconds at 55° C. and 60 seconds at 72° C. PCR was terminated after a 10 minutes elongation step at 72° C.

Gene Nomenclature

The ATM gene is mapped to chromosome 11q22.3, and the target sequence used was obtained from GenBank, accession no. U82828. 1. The target sequence was located within intron 38 and exon 39. The polymorphisms examined correspond to NCBI SNP reference numbers rs3092829 (IVS38-8 T/C), rs1801516 (5557 G/A), and rs1801673 (5558 A/T).

Cycling Temperature Capillary Electrophoresis

A standard 96-capillary DNA sequencing instrument MegaBACE 1000 DNA Analysis System (Amersham Biosciences, Oslo, Norway) was used for cycling temperature capillary electrophoresis (CTCE). Coated fused silica capillaries were obtained in sets of 16 capillaries (Amersham). The capillaries have an outer diameter of 200 μm and an internal diameter of 75 μm. The distance from the cathode to the detector is 40 cm. The MegaBACE platform uses a scanning cofocal laser for detection of fluorescence in each capillary. Standard sequencing 3% linear polyacrylamide with urea was replaced prior to every run, by applying high-pressure nitrogen gas. PCR products were diluted 1:20 in water prior to electrokinetic injection, accomplished by applying 200 V/cm for 12 s. Electrophoresis was performed at a constant 150 V/cm. The partial melting of DNA fragments was achieved by a combination of urea in the matrix and decreasing cycling temperature going from 48° C. to 46° C. in 1° C. steps. Each temperature was held for 30 seconds, and the gradient was repeated 15 times and followed by a hold at 44° C. until the end of the run. With the present setup 96 samples were analyzed within 40 minutes by use of CTCE.

DNA Sequencing

All samples with aberrant peak patterns compared to the internal standard were sequenced. The unlabeled sequencing primer for the forward sequence was a primer based on the first 20 bases in the GC-clamp (5′ GCG GGC GGC GCG GGG CGC GG 3′; SEQ ID NO:15) and the same 20-mer primer used for PCR described above was used to generate the confirmatory reverse sequence. Samples were sequenced using the MegaBACE standard protocol and sequencing analysis was performed on the same instrument under sequencing conditions as described by the manufacturer (Amersham).

Thermodynamics

All microhaplotype combinations of the three polymorphisms with nucleotide number IVS38-8 T/C, 5557 G/A, and 5558 A/T were simulated using DNA melting software WinMelt (MedProbe) based on the Poland algorithm and variants thereof. Normalization of microhaplotype melting behavior relative to the wild type (e.g., the most frequent allele combination) in the nucleotide positions IVS38-8T-5557G-5558A was calculated using the following formula:

$$\sum \text{?} ( ( 1 - \frac{{Y}_{1}}{{X}_{1}} ) \times 100 )$$

$$\text{?} \text{indicates text missing or illegible when filed}$$

where X and Y are estimated melting temperatures for wild type and microhaplotype at given base pair (n), respectively.
Statistical methods

The two-tailed χ² test was used to calculate statistically significant (P<0.05) differences in allele, genotype, and microhaplotype frequencies between the normal population and the rectal cancer group.

Results

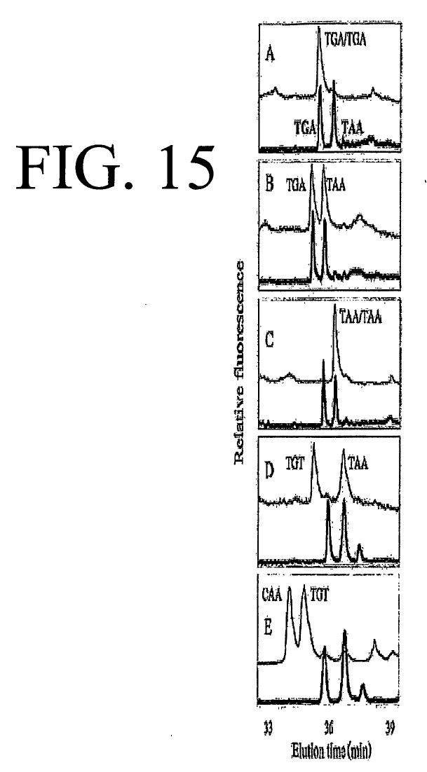

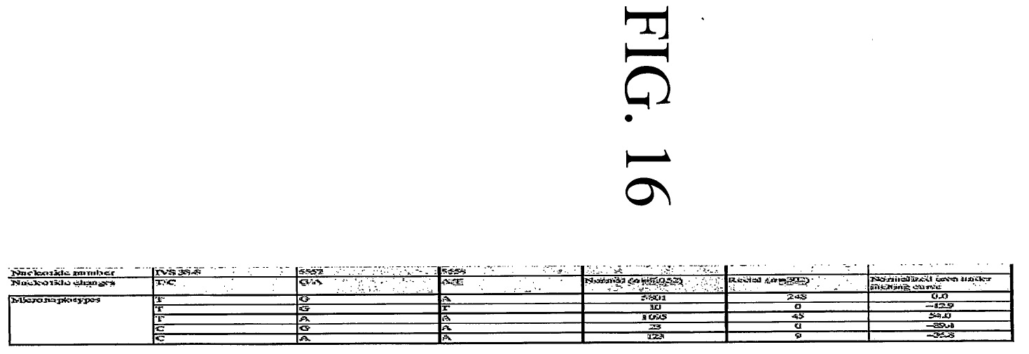

Three ATM polymorphisms, IVS38-8 T/C, 5557 G/A, and 5558 A/T were analyzed in 3,526 blood donors and 151 sporadic rectal cancer by CTCE. Alleles were resolved by cooperative melting of double strand DNA. Partial melting of DNA fragments was detected as change in mobility during electrophoresis. FIG. 15 displays five genotypes of the three SNPs examined. The first three samples (A, B, C in FIG. 15) labeled with 6-FAM coeluted with the respective alleles of the TAMRA-labeled internal standard. FIGS. 15D and 15E display two electropherograms with alleles that did not coelute with the internal standard. Theoretical melting behavior of each microhaplotype was normalized to the wild-type IVS38-8 T 5557 G 5558 A (FIG. 16). Microhaplotype IVS38-8 T 5557 A 5558 A has positive value and eluted after the wild-type (IVS38-8 T 5557 G and 5558 A). The remaining microhaplotypes have negative values (FIG. 16), which indicate higher temperature properties as compared to the wild-type. Consequently these alleles elute before the wild-type (FIGS. 15D and 15E). Knowledge of melting behavior for different microhaplotypes relative to the wild-type in combination with DNA sequencing allowed direct determination of microhaplotypes. The electropherogram in FIG. 15D shows that one peak coeluted with the IVS38-8 T 25 5557 A 5558 A peak in the standard and one peak eluting before the standard. Sequencing this sample revealed heterozygosity in position 5557 G/A and 5558 A/T (data not shown). This information in combination with normalized melting values (FIG. 16) made it possible to identify the microhaplotypes as IVS38-8 T 5557 G 5558 T and IVS38-8 T 5557 A 5558 A. The electropherogram in FIG. 15E shows that both peaks eluted before the standard. This indicates that the sample has a higher melting temperature than the wild-type. Sequencing verified a triple heterozygous sample for the three SNPs (data not shown). Consequently the microhaplotypes of this sample must be IVS38-8 C 5557 A 5558 A and IVS38-8 T 5557 G 5558 T.

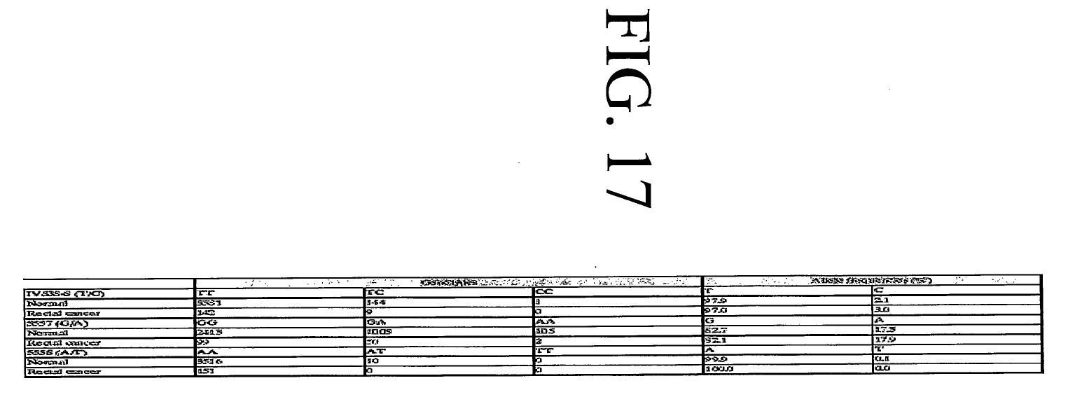

The observed genotype frequencies of the three examined ATM SNPs (IVS38-8 T/C, 5557 G/A and 5558 A/T) and the frequencies of the alleles are shown in FIG. 17. The genotypes of the three polymorphisms were in Hardy-Weinberg equilibrium for both groups (data not shown).

For SNP IVS38-8 there were no patients found in the sporadic rectal cancer group with the genotype combination CC. For SNP 5558 the combinations A/T and TT were not found in the cancer group, and TT was not found in the normal population. No statistically significant differences between the two groups were found for the SNPs, IVS38-8 T/C (P=0.52), 5557 G/A (P=0.28), and 5558 A/T (P=0.51). The allele frequencies in the three SNPs were also statistically insignificant (IVS38-8 T/C, P=0.49; 5557 G/A, P=0.78; 5558 A/T, P=0.51).

The frequencies of the microhaplotypes are shown in FIG. 16. The observed frequencies of the microhaplotypes were in good correspondence with estimated microhaplotypes based on allele frequencies (data not shown). In the 7,052 alleles analyzed in the normal population there were three microhaplotypes that were not found and two of these microhaplotypes consisted of ATT, in codon 1853, which codes for isoleucine. In the sporadic rectal cancer group with 302 alleles only three microhaplotypes were found. No statistically significant differences in the frequency of microhaplotype were found within the two groups (P=0.40).

Discussion

The results described herein, obtained by using a novel method for identifying and quantifying polymorphisms and allele frequencies in a population, demonstrate no correlation between incidence of rectal cancer and the three ATM polymorphisms IVS38-8 T/C, 5557 G/A, and 5558 A/T and microhaplotypes. Although no association was found between selected SNPs and rectal cancer, the method used to analyze SNPs, genotypes, or microhaplotypes exhibits a high throughput, sensitivity, and robustness for DNA variation analysis.

### EXAMPLE 5

### Evaluation of Denaturing Conditions in Analysis of DNA Variants Applied to Multi-capillary Electrophoresis Instruments

Denaturant slab gel techniques (DGGE and variants thereof) and denaturing CE (DCE) have been used for the analysis of mutations and single nucleotide polymorphisms by several research groups. DCE applied to commercially available capillary electrophoresis instruments has been demonstrated to be sensitive, specific, and robust for mutation and SNP analysis. However, instruments currently available on the market are not designed for DCE in their present forms. In both single and multi-capillary instruments inaccurate temperature control units lead to irreproducible denaturing conditions, both between runs and from capillary to capillary within an electrophoresis run. Cycling a temperature gradient several times around a theoretically defined optimum for a given DNA fragment results in more reproducible and controllable denaturing conditions compared to a constant denaturant or single sweep temperature gradient. In this example, it is demonstrate that with the temperature differences observed, the common technique of applying a temperature gradient is not sufficient for optimal separation conditions in all capillaries. Furthermore, it is demonstrated that cycling of the temperature around a fragment's theoretically calculated melting temperature during electrophoresis leads to reproducible results in all capillaries. These results clearly show the potential for standard commercial capillary instruments in automated cycling temperature CE.

Constant Denaturant Capillary Electrophoresis (CDCE), introduced in 1994 (Khrapko, K. et al, 1994, Nucleic Acids Res., 22:364-369) has been demonstrated to have high sensitivity as a method in mutation detection. CDCE was performed on home-built apparatus and was based on the same separation principles as its slab gel analogs, Denaturant Gradient Gel Electrophoresis and Constant Denaturant Gel Electrophoresis (Hovig, E. et al., 1991, Mutat. Res., 262:63-71). The separation principle utilized by these methods is based on differential melting of DNA (Fischer, S. and Lerman, L., 1980, Proc. Natl. Acad. Sci. USA, 77:4420-4424; Fischer, S. and Lerman, L., 1979, Cell, 16:191-200; Fixman, M. and Freire, J., 1977, Biopolymers, 16:2693-2704). In short, mutated sequences can be separated from wild-type sequences on the basis of nucleotide sequence and length. A double-stranded fragment undergoes melting into discrete single strand domains when exposed to denaturing temperature or a chemical denaturant (e. g., urea or formamide). Under properly selected partially denaturing conditions, different DNA variants will exhibit alternative secondary structures that will allow their electrophoretic separation in a sieving electrophoresis matrix. The separation performance can be enhanced further if the two strands of the DNA fragment are forced to stay in proximity to one another during the partial denaturing process. This can be easily achieved by extending the fragments at one end by a high-melting domain, for example an artificial GC-rich sequence (GC-clamp) (Myers, R. et al., Nucleic Acids Res., 13:3111-3129). The melting temperature of the clamp is designed to be above the optimum partial-melting temperature, therefore holding the two strands together at one end.

Over the past several years, CDCE has been used for detection of various low-level frequency mutations. Recently, we have demonstrated the use of semiautomated mutation analysis of KRAS using the CDCE technique on commercial single and multi-capillary instruments. In those studies, the separation temperature was accurately maintained to achieve high resolution. In the multi-capillary CDCE format, a home-made solid-state heater accommodating all 96 capillaries was necessary to maintain constant separation temperature. With an unmodified apparatus, the temperature in the capillary chamber could not be maintained stable enough to permit uniform denaturing conditions in all capillaries.

The main purpose of the present study was to demonstrate that neither constant temperature, nor single temperature gradients, allow optimal temperature distribution across all capillaries in a 96-capillary format. To eliminate this effect, we demonstrated that cycling of the separation temperature around the theoretically calculated thermodynamic properties of the relevant DNA fragment during electrophoresis allowed reproducible results in all capillaries and over repeated runs.

DNA and PCR

PCR samples with target sequences in KRAS exon 1 and Lymphotoxin alpha promoter were used to demonstrate separation in different capillaries under denaturing CE. Primers and PCR conditions for the fragments used have been described previously (see Examples 1 and 2). Both the KRAS and Lymphotoxin alpha PCR products contain one low melting domain covering the target sequence and one artificial high melting domain.

An internal standard with five peaks was created by mixing labeled PCR products from three homozygous mutant samples (a codon 12 GGT to TGT mutant, a codon 12 GGT to CGT mutant and a codon 13 GGC to AGC mutant) with unlabeled wild-type PCR product. Two of the heteroduplex variants co-migrated in this standard.

Theoretical Thermodynamics

Theoretical melting profiles of the different target fragments were obtained from the Poland algorithm and by using the WinMelt computer program, applying the Melt87 program (MedProbe, Oslo, Norway). The optimal separation temperature proposed by the WinMelt/Poland Web page was adjusted on the basis of the concentration of urea in the matrix. For each molar increment of urea concentration the temperature was lowered by approximately 3 K.

CE

PCR samples were analyzed in a standard 96-capillary DNA analyzer (MegaBACE™ 1000 DNA Analysis System, Amersham Biosciences, Sunnyvale, Calif., USA). The distance from the anode to the detector was 40 cm. Linear polyacrylamide (MegaBACE LPA) containing 7 M urea was replaced in capillaries prior to each run. Standard coated capillaries were used. PCR products were loaded into the capillaries by electrokinetic injection at 200 V/cm for 12 s. The electrophoresis was carried out at a constant field of 150 V/cm. All samples were analyzed in 96-well plates. Laser-induced fluorescence was used with excitation at 488 nm (blue laser) and detection of emission at 520 nm (FAM channel).

Denaturing Conditions and Temperature Control

For each run, PCR products from the same target sequence were dispensed to all wells in a 96-well plate and analyzed under various denaturing conditions. The denaturing temperature in the capillary chamber was either held constant at theoretically calculated optimal value or cycled for varied time intervals around the calculated optimal temperature. The denaturing conditions, e.g., the temperature profiles, were controlled through the Instrument Control Manager (ICM) software package (Amersham Biosciences). Temperature sensors were placed in close proximity to the capillaries. All sensors were placed at the same vertical level, and at different horizontal locations. One temperature sensor was placed at the front part of the capillaries (A), one in the middle part (B), and one at the back of the capillaries (C). The temperature recording from the three thermocouple probes (K type) (Sievert Max, Oslo, Norway) was performed using an analog computer board (IOtech, Inc., Cleveland, Ohio, USA). The temperature probes were calibrated before the temperature recording took place, and the rate of measurement was one point per second(1 Hz).

Results

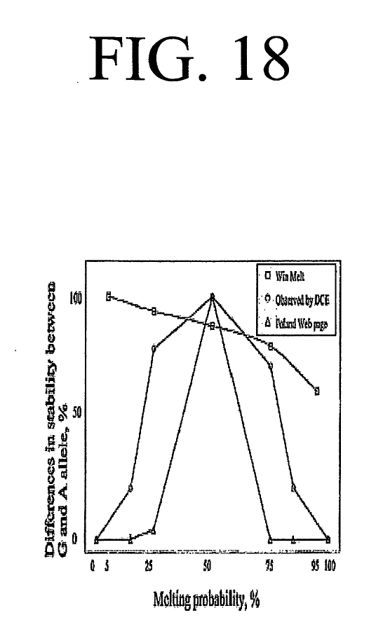

The first step required in a capillary electrophoresis protocol using differential melting on a defined target fragment, is the determination of the optimal melting conditions for the separation of homozygous alleles as given by one of several available computer programs utilizing variants of the Poland algorithm. These programs include the Poland algorithm, and the computer software program WinMelt/MacMelt designed to calculate thermodynamics of double stranded DNA. FIG. 18 demonstrates the calculated relative stability of two alleles of the lymphotoxin alpha fragment at different melting probabilities, and the experimentally observed difference in migration (e. g., scan number variant 1 minus scan number variant 2). For the experimental data, the highest temperature not causing branching of the double stranded DNA was defined as 0% difference in stability between the alleles. The lowest temperature with complete branching of the low melting domain was defined as 100%. At these temperatures no allele separation was expected. Note that the thermodynamics calculated using the Poland web page (H) predicted the observed optimal thermodynamic separation (9), as compared to the actual behavior of DCE analysis of the fragments. WinMelt (F), however, did not predict the presence of a specific optimal thermodynamic separation region between the fragments. Thus, quite large differences in thermodynamics were predicted at all melting probabilities with this program.

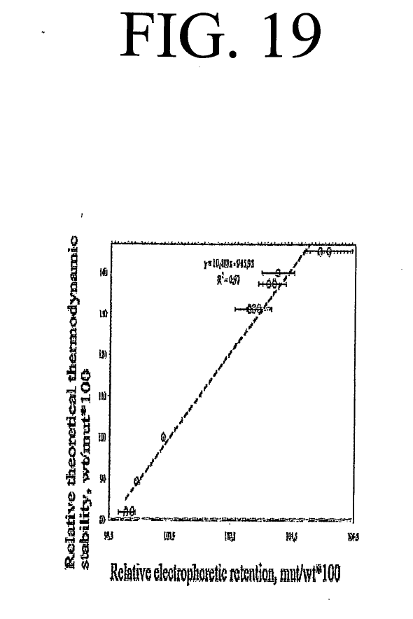

The thermodynamics (differences for each base pair at 50% melting probability) of all different oncogenic mutants in KRAS exon 1 were calculated with WinMelt at 50% melting probability. Thereafter, the calculated values were correlated with observed migration differences for each mutant. A linear correlation (r₂=0.97) was found between migration times of the different variants and the theoretical thermodynamics calculated (FIG. 19). The same exercise was performed using theoretical melting probabilities obtained from the Poland web page. A linear relationship found was r²=0.93.

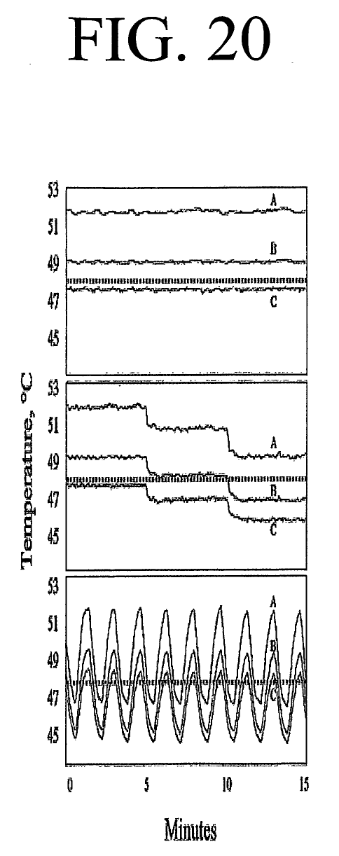

To be able to transfer the calculated melting properties of a fragment directly to CE analysis, the temperature distribution in the capillary chamber was examined. External temperature sensors were placed inside the capillary chamber of a MegaBACE™ 1000 in close proximity to the capillaries. Several temperature profiles were constructed to give the same average denaturing temperature (48° C.) in the capillary chamber. The results showing local temperature differences within the capillary chamber in these experiments are presented in FIG. 20.

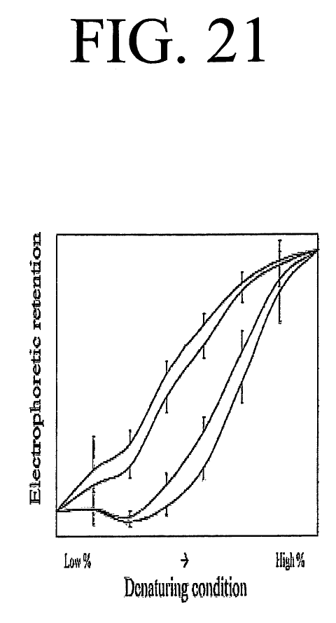

Since the retardation of the different DNA variants in the sieving matrix is a gradual process, separation of some homoduplexes and heteroduplexes may be achieved even outside the optimal denaturing conditions. The four lines in FIG. 21 demonstrate the observed migration (scan number) of two homoduplexes and two heteroduplexes in heterozygous lymphotoxin alpha samples (n=11) analyzed at six different denaturing temperature conditions. The two lower lines represent the homoduplexes and the two upper lines the heteroduplexes. At low denaturation, separation of the two heteroduplexes is achieved, as the heteroduplexes have thermodynamics with a lower stability than the homoduplexes. The standard deviation (e.g., variation in scan number) caused by the local differences in temperature in the capillary chamber is depicted in FIG. 21. The result resembles the “S” shape observed in perpendicular DGGE.

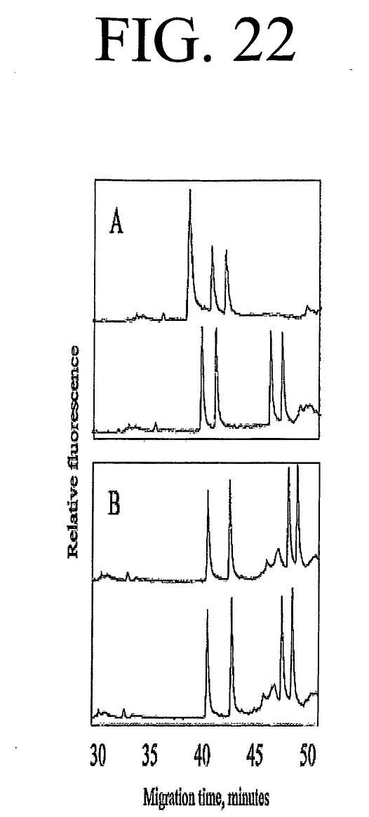

The target lymphotoxin alpha samples were then analyzed under different denaturing conditions. FIG. 22A shows two electropherograms of the same sample, analyzed in the same run and in different capillaries under constant denaturant conditions. Since the capillaries are exposed to different temperatures (i. e., local temperature variation in the capillary chamber, see FIG. 20), the separation of homoduplexes and heteroduplexes differs in the two capillaries. Such capillary to capillary irreproducibility clearly prevents usage of a commercial capillary array system for CDCE type analysis. The PCR fragments shown in FIG. 22B were analyzed under cyclic temperature conditions, from 48° C. to 52° C. in 15 cycles. The delay at each boundary temperature was set to 30 seconds. With the cycling denaturing conditions, identical separation patterns were repeatedly obtained in different capillaries.

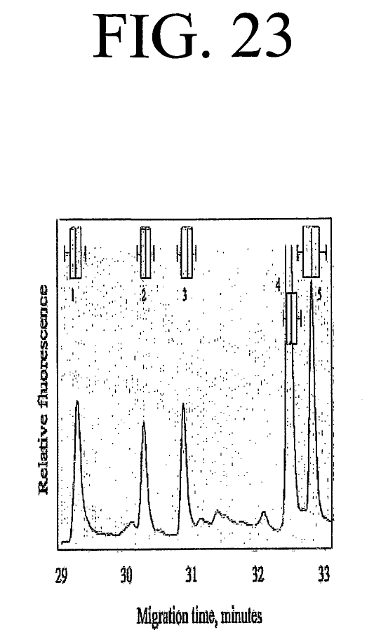

The reproducibility of cycling temperature capillary electrophoresis (CTCE) was evaluated by measuring the migration times of all peaks in a KRAS construct standard (see Examples 1 and 2). Separation of standard peaks and box plots, representing 649 samples standardized to migration time, is illustrated in FIG. 23. A normalized migration time was calculated by dividing peak migration time (i.e., the scan number) by the sum of all migration times of all peaks in the same capillary.

Discussion

Capillary electrophoresis on multi-capillary instruments, with the use of partial denaturing conditions, represents a recent approach in DNA variation analysis. Although this method is based on an established melting gel theory, optimization of denaturing conditions has generally been necessary by computer prediction and experimental verification for each individual target sequence. Described herein is the evaluation of the use of alternative temperature control strategies in order to achieve optimal denaturing conditions for analysis on a standard commercial 96 capillary instrument. PCR fragments, previously evaluated by different melting techniques were used for analysis.

The melting characteristics of the target sequences were first calculated by the Poland algorithm. The resulting theoretical thermodynamic behavior of the fragments was compared to experimentally observed migration of homoduplex forms of each fragment. An acceptable correlation was found between the calculated 50% melting probability values for the simulation with respect to migration differences between the two separated alleles. Consequently, melting algorithms developed for use in DGGE could directly be utilized on the capillary platform. However, at probability levels other than 50%, the calculated values were not in agreement with experimental results for the different homoduplex variants. The Poland web page has an option for estimating fragments containing mismatches (i.e., heteroduplexes). These theoretical simulations did not correspond well with the observed retention of heteroduplexes by cycling temperature capillary electrophoresis (data not shown). Apparently, theoretical calculation of thermodynamics of heteroduplexes in capillary melting techniques and its correlation with experimental data needs further investigation.

By the use of WinMelt, we calculated the 50% melting probability of all oncogenic mutations in KRAS exon 1, and a linear correlation was found between relative electrophoresis retention of fragments and the relative thermodynamic stability. This correlation was used to determine the relative migration of a variant fragment prior to the actual experiment. Based on the above thermodynamic calculation, the relative positions of mutant homoduplexes when related to wild-type could also be predicted. The relative position of a homoduplex in an electropherogram could then be used for direct identification of the variant from a set of possible sequences. This been demonstrated with KRAS exon 1.

A critical parameter for denaturing CE with multiple capillaries is the local temperature fluctuations within the capillary chamber. As shown in FIG. 20, differences across different capillaries were measured with both constant temperature and a single-sweep temperature gradient. As a consequence of inhomogeneous temperature, loss of separation was observed in some capillaries (FIG. 22). The main reason for these fluctuations was probably a non-equal air circulation inside the capillary chamber. As a consequence of the inaccurate temperature control, an attempt to equilibrate the differences by introducing temperature gradients and temperature cycling profiles was made.

It was expected that in order to assure equivalent conditions for all separated samples, the gradient should be symmetrical, i.e., samples should spend equal time at each temperature. Fast cycling should therefore better serve separation reproducibility among parallels. During construction of different temperature cycling profiles it was observed that the maximum achievable temperature ramp rate was approximately 0.1 K/s. By ramping the temperature between maximum and minimum temperatures in the cycles and taking into account the cycling time, smooth sinus-like temperature profiles were achieved. The maximum rate of cycling was then obtained from the temperature range and the temperature ramp rate. It was observed that too high cycling of temperature resulted in lower temperature amplitudes in the sinus curve. A maximum of 20 cycles spanning 3 K in 20 min yielded reproducible temperature curves. Secondly, another notable advantage of fast cycling is the elimination of problems due to changes in the optical response of the instrument. Most commercial instruments include precise optical elements (filters, lenses, pinholes, mounts, holders, etc.) that are susceptible to temperature changes. The usual impact of changing temperature on optics includes loss of alignment and focus, resulting in reduction of signal-to-noise. With fast temperature cycling, most optical elements, which are made of solid materials, do not follow the rapid changes. As a result, their temperature is equilibrated at an average constant temperature and maintained during the entire run. The reproducible performance of the instrument is therefore maintained during the temperature gradient operation. Thirdly, use of a cycling gradient further allows significant increase in sample throughput by application of multiple-injection workflow (Example 1). Most commercial capillary array instruments allow controlled temperature during the run, thus this approach of DNA variation analysis should not be instrument dependent.

The use of denaturant gradients with Joule heating and temperature zone have been applied to single capillary instruments (Gelfi, C. et al., 1996, BioTechniques, 21:926-932; Zhu, L. et al., 2001, Electrophoresis, 22:3683-3687; Schell, J. et al., 1999, Electrophoresis, 20:2864-2869). However, these strategies did not improve the sensitivity and specificity of the mutation analysis as compared to CDCE (Khrapko, K. et al., Nucleic Acids Res., 22:364-369; Gelfi, C. et al., 1997, Electrophoresis, 18:724731). In the present setup, the use of gradients and cycling in the multi-capillary system was adapted for an entirely different reason: The original purpose of the gradient was to eliminate local temperature differences in the capillary chamber. Unexpectedly, improved separation in each capillary was observed (FIGS. 22 and 23). However, the benefit of denaturing gradients applied to slab gel techniques (TGGE, TTGE, and DGGE), with regard to robustness, appeared as a secondary effect of a cycled temperature gradient. In both gradient slab gels and CTCE, fine-tuning of denaturant conditions is not needed, as is the case in CDGE and CDCE. Sensitivity and specificity reported for CDCE is preserved in the CTCE setup. This is because the separation time in CTCE (i.e., the time each capillary spends at the optimal denaturing temperature) is comparably much longer than for single gradients.

In a common configuration of DGGE, analysis is performed on a slab gel with defined gradient of chemical denaturant perpendicular to the electrophoretic direction. The resulting “s-shaped” diagram is used to locate optimal denaturing conditions. In a capillary format, a similar analysis can be performed by comparing separation profiles of samples separated at different denaturing conditions. The results presented in FIG. 21 provide information on the temperature interval in which separation of homoduplexes and heteroduplexes is achieved. Furthermore, this can be used in settings were different target fragments are to be analyzed in the same run. By adopting a compromise with regard to the denaturing conditions for individual fragments, samples differing by up to 6 K in thermodynamics of the low melting domain could be analyzed in parallel capillaries (data not shown).

Additionally, running the fragment under multiple different denaturing conditions helps to discriminate artifact peaks formed during PCR or spikes in electrophoresis. Their migration will not be significantly altered by small changes in denaturing conditions, and can thus be excluded as aberrant (Bjørheim, J. et al., 2002, J Sep. Sci., 25:637-647).

To demonstrate reproducibility of the temperature cycling, 7 subsequent runs of an artificial KRAS construct exhibiting 5 peaks (representing 5 different KRAS exon 1 variants) were performed. The migration times of all peaks were normalized to compensate for offset in individual traces (e.g., due to differences in electrophoretic current). Variance of the normalized migration times is shown in FIG. 23. The reproducibility of the method is demonstrated by the mean and standard deviation in the box plot transposed over a representative electropherogram of the KRAS construct. It can be clearly seen from this figure that the individual boxes are well-spaced. This indicates a complete baseline resolution of all peaks at any given combination of runs or capillaries.

Denaturant capillary electrophoresis has previously been demonstrated to have high sensitivity and high throughput potential in DNA variation analysis. With CDCE, time-consuming optimization has to be expected as well as lower reproducibility compared to cycling of temperature in denaturant CE. Consequently, mutations in target sequences have been detected, but their identity had to be revealed by other means (Guldberg, P. and Guttler, F., 1993, Nucleic Acids Res., 21:2261-2262). Analysis with cycling temperature exhibits a high robustness and reproducibility allowing direct identification of mutations based on peak migration times in electropherograms. The results presented in this study can be directly transferred to any multi-capillary sequencing instrument with air-ventilated chamber without any need for hardware changes. In theory, all fragments previously analyzed with denaturing slab gel techniques can be analyzed with cycling temperature in a denaturant capillary electrophoresis setup without any need for complex PCR procedures. In conclusion, the theory of well-known denaturant slab gel techniques can be transferred to the multi-capillary format applied to denaturing CE. This opens a possibility for filly automated analysis of DNA variation in large sample sets for high throughput screening.

### EXAMPLE 6

### Simultaneous Allele Separation of DNA Variants in Twelve Different Fragments, with Large Differences in Thermodynamics, by High Throughput Cycling Temperature Capillary Electrophoresis.

Described herein is an analytical tool for parallel detection and screening of nucleotide variation (mutations and single nucleotide polymorphisms) in DNA fragments. Twelve different alleles with known single base DNA variation sited in eleven PCR fragments with theoretical melting temperature ranging from 69 to 78° C. were amplified in 96-well plates by a common protocol. Following PCR, separations of alleles were achieved by cycling temperature capillary electrophoresis (CTCE) in a standard DNA sequencing instrument. By applying denaturing temperature in cycles, covering the theoretical melting temperatures of all fragments, separation between variants and canonical alleles were achieved in all samples in parallel runs. Smaller temperature cycles resulted in loss of separation between homoduplexes in fragments with theoretical melting temperatures not included in the cycling temperature gradient. On the other hand, separation between variants was greater with more focused cycling gradients than with a general large temperature cycle gradient. By adopting a compromise with regard to the denaturing conditions for individual fragments, samples with large difference in thermodynamics were analyzed in parallel capillaries with CTCE. The method opens a possibility for one step detection and screening of DNA variants in 96 different fragments without need for individual analysis design and optimization.

By introducing large temperature gradients, separation of DNA variants with large differences in thermodynamics is achieved by CTCE. This allows analysis of different DNA fragments in the same run. The analysis is performed in a 96 capillary instrument with four channels, allowing simultaneous analysis of up to 384 different samples within 40 minutes. Further, the fragments can be scanned for unknown DNA variation as previously reported for melting gel techniques. For demonstration purposes, twelve DNA variants with 9 K difference in thermodynamics were analyzed under various denaturing conditions in an automated and high throughput system with the aim of separation all variants in parallel runs.

DNA Samples and PCR

Genomic DNA was extracted from anonymous blood donor samples by standard DNA extraction kit (Qiagen Inc., Valencia, Calif., USA). Primers were designed with Primer3 (Whitehead Institute, Cambridge, Mass.) software for the target sequences, using standardized parameters, thus all fragments could be amplified with the same PCR conditions. Twelve different samples were analyzed in replicates of eight (12 DNA fragments×8 wells=96 wells) in 96-well plates. Amplification was performed on a PTC-200 thermal cycler (MJ research, Waltham, Mass., USA), by mixing 50 ng genomic DNA with 25 μM of each dNTP (Perlin Elmer, Oslo, Norway), 10× Taq buffer, 1 unit Taq and 5 pmol of each primer (MedProbe, Oslo, Norway) in a final volume of 25 μL. The cycling parameters were 35 cycles of denaturation for 30 seconds at 94° C., annealing at 56° C. for 30 seconds and elongation at 72° C. for 1 minute, followed by elongation step of 72° C. for 10 minutes.

Target Sequence Identification

Fragments were design for analysis of DNA variation identified by NCBI SNP reference number rs, 1) Breast Cancer, Type 1 (BRCA 1) rs799923, 2) BRCA 1 rs16940, 3) 5,10-Methylenetetrahydrofolate Reductase (MTHFR) rs1801133, 4) Opsin cone pigments (OPN1) (base variation between long and medium wave genes at base pair 78173 in Genbank sequence AC092402), 5) MTHFR rs1801131, 6) MTHFR rs2274976, 7) Cystathionine-Beta-Synthase (CBS) rs234706, 8) Nad(P)H Dehydrogenase, Quinone 1 (NQO1) rs1800566, 9) Dihydropyrimidine Dehydrogenase (DPYD) rs3918290, 10) DPYD (-10 base pair from rs3918290), 11) DPYD rs1801265, 12) Cytotoxic T Lymphocyte-Associated 4 (CTLA-4) rs5742909.

Theoretical Thermodynamics

Theoretical melting profiles of the different target fragments were obtained by applying Poland algorithm (Poland, D., 1974, Biopolymers, 13:1859-1871) through the use of WinMelt computer program (MedProbe, Oslo, Norway). The optimal separation temperature proposed by these programs was adjusted on the basis of the urea concentration in the matrix. For each molar increment of urea, the temperature was lowered approximately 3.2 K (Fodde, R. and Losekoot, M., 1994, Hum. Mutat., 3:83-94).

CE

PCR samples were analyzed in a standard 96-capillary DNA analyzer (MegaBACE™ 1000 DNA Analysis System, Amersham Bioscience, Sunnyvale, Calif., USA). The distance from the anode to the detector was 40 cm. Linear polyacrylamide (MegaBACE LPA) containing 7 M urea was replaced in capillaries prior to each run. Standard coated capillaries were used. PCR products were loaded into the capillaries by electro kinetic injection at 200 V/cm for 12 seconds. The electrophoresis was carried out at a constant field of 200 V/cm. Laser-induced fluorescence was used with excitation at 488 nm (blue laser) and detection of emission at 520 nm (FAM channel).

Denaturing Conditions and Temperature Control

In each run, a 96-well plate with the same distribution of fragments was analyzed under various denaturing conditions. The denaturing temperature in the capillary chamber was either cycled with large amplitudes covering the theoretical thermodynamics of all selected fragments (13 K) or with smaller amplitudes down to 3 K. After the cycling, the temperature in the chamber was reduced to 40° C. The denaturing conditions, e.g., the temperature profiles, were controlled through the Instrument Control Manager (ICM) software package (Amersham Bioscience). The MegaBACE™ Sequence Analyzer View and Edit software program (Amersham Bioscience) was used to measurer the migration times of all peaks. Three calibrated temperature sensors were placed in close proximity to the capillaries at the same vertical level, but at different horizontal locations. One temperature sensor was placed in the front part, one in the middle part and one at the back of the capillaries. The temperature recording from the three thermocouple probes (K type, Sivert Max, Oslo, Norway) was recorded using an analogue computer board (Iotech, Inc., Cleveland, Ohio, USA) at a rate of 1 Hz.

Results and Discussion

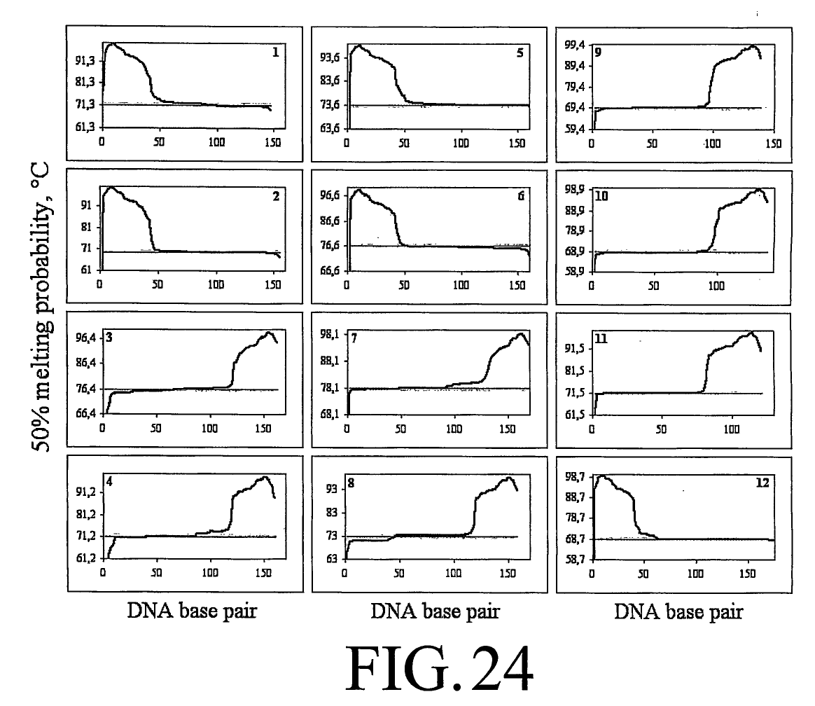

Theoretical melting profile analyses of the selected target fragments with attached GC-clamp were first performed. The thermodynamics of the canonical DNA sequences of all twelve fragments are illustrated in FIG. 24. The thermodynamics of the low melting domain of the CTLA-4 sequence showed a theoretical melting temperature, 68.7° C. The selected sequence from the CBS gene had the highest thermodynamics of the low melting domain, 78.1° C. All other fragments had theoretical melting temperatures between these extremes. Variant sequences were also analyzed and demonstrated a theoretical difference in thermodynamics as compared to wild-type sequences (data not shown). The selected fragments thermodynamics are a representative selection of amplicons designed for melting gel analysis. On average, fragments designed on random DNA will display thermodynamics between 70-75° C. This is exemplified by computing the thermodynamics of a random 20000 base pair in chromosome 13. The average thermodynamic was found to be 68.9° C. per base pair, with iso-melting domains in the range of 100 to 200 base pairs. Thus, theoretically, 96 different fragments can be constructed with thermodynamics similar to that of the fragments used in this study.

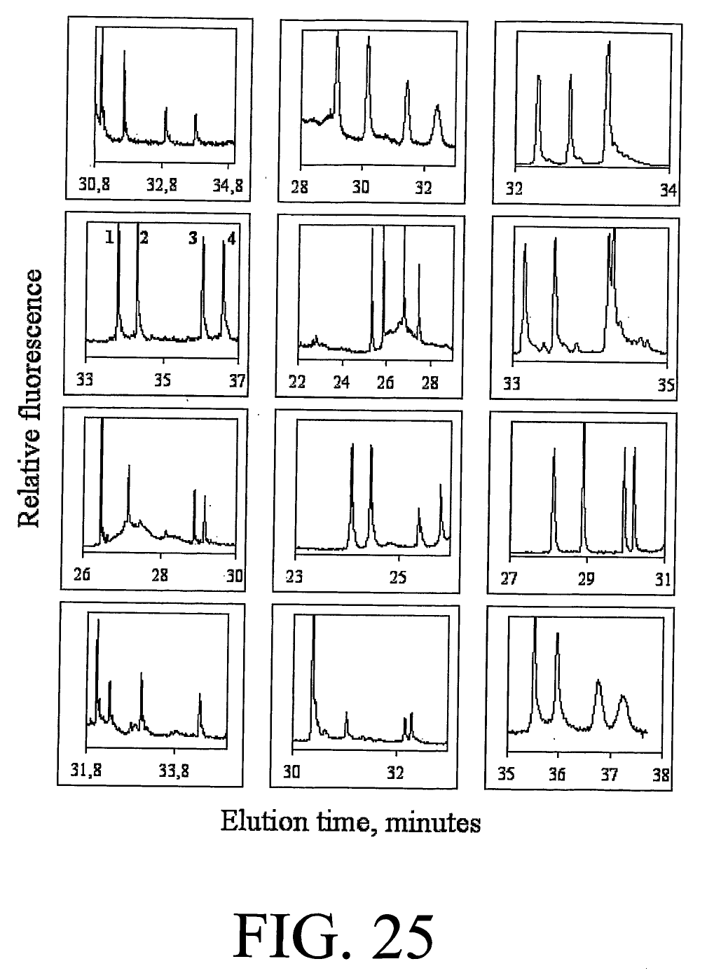

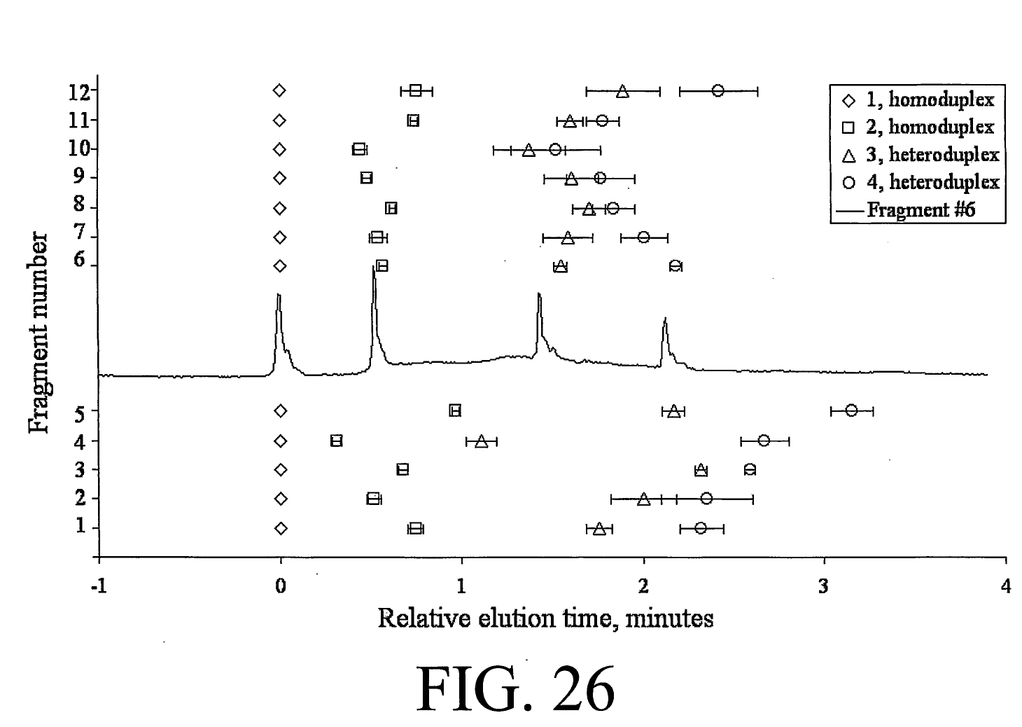

After evaluation of the thermodynamics, creation of a 96-well plate with replicates (n=8), of the different PCR amplified fragments were electrophoresed under various denaturing conditions. When temperature in the capillary chamber was cycled between 59-47° C. (a denaturing temperature interval that covers the thermodynamics of all of the fragments) five times, separation of all twelve fragments were achieved repeatedly in parallel runs and in different capillaries (FIG. 25). Separation of homoduplexes and heteroduplexes was performed within 40 minutes for all samples. As seen by the base line separation between the homoduplexes (peak 1 and 2) variant homozygous samples can directly scored in electropherograms by use of an internal standard, with a different label (e.g., TAMRA). This is in clear contrast to methods like heteroduplex analysis (HA) and dBPLC, where separation of the homoduplex species has not been reported, except for large deletion/insertions (Cremonesi, L. et al., 2003, Br. J Haematol., 121:173-179; Kozlowski, P. and Krzyzosiak, W., 2001, Nucleic Acids Res., 29:E71). The migration times of all peaks of all fragments in a 96-well plate analyzed with the above-described denaturing conditions were measured. The values found for the second homoduplex and heteroduplexes were normalized to the migration of the first homoduplex peak. In FIG. 26 the mean migration times of peak maximum for the homoduplex and heteroduplex peaks is shown. The brackets indicate one standard error of the mean in FIG. 26. Baseline separation of all homoduplexes was achieved under these cycling conditions. Occasionally, some of the heteroduplex peaks co-migrated with the large temperature gradient, due to complete melting of the low melting domain of both heteroduplexes. Incorporated in the figure is an electropherogram presenting analysis of fragment number 6. This is the first time that separation of twelve different alleles has been achieved to such an extent that direct genotyping is possible. Thus, comparing this method with exciting DNA variation discovery methods, dHPLC, HA or SSCP renders this adaptation of DGGE on to a regular capillary DNA sequencing instrument the most powerful tool in DNA discovery.

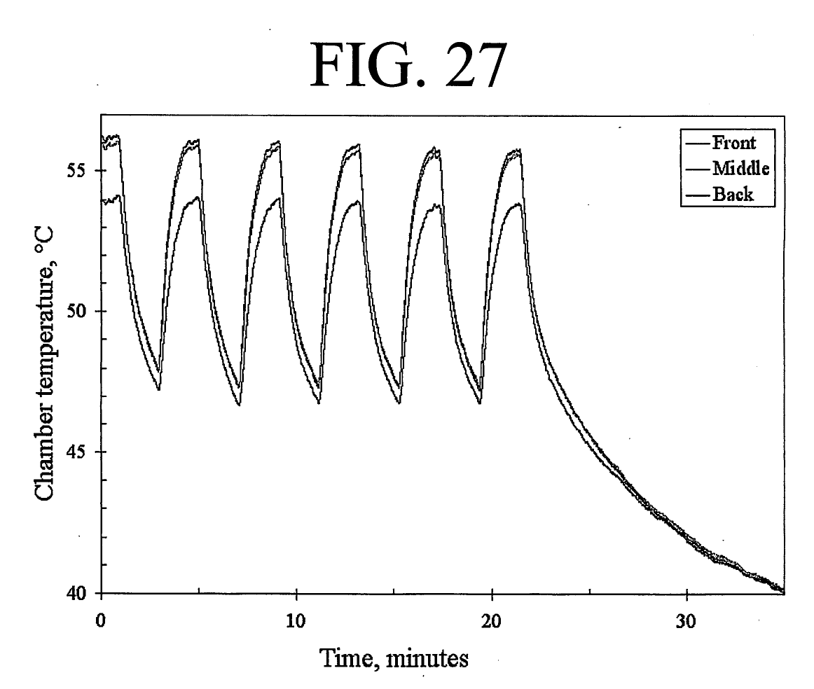

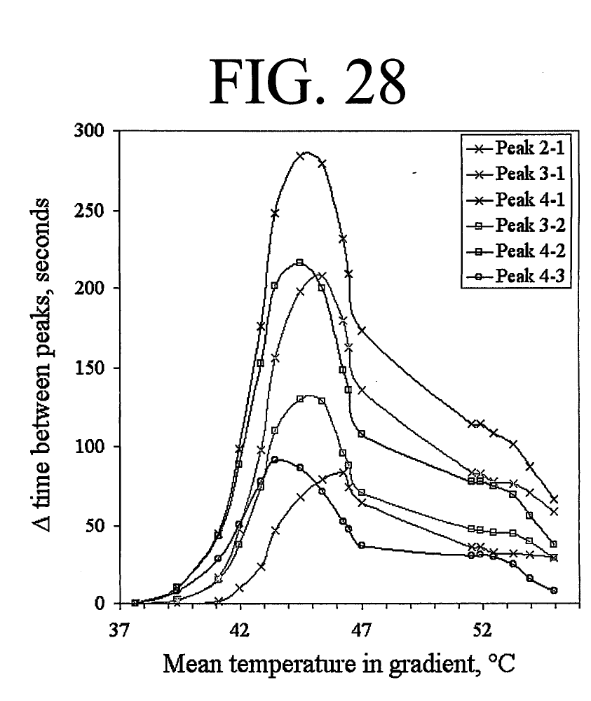

Constant denaturing capillary electrophoresis results were dependent on very precise temperature control to be able to reproducibly separate alleles (Li-Sucholeiki, X. et al., 1999, Electrophoresis, 20:1224-1232). With this in mind, measurement of the denaturing temperature at different locations within the capillary chamber was performed during electrophoresis. FIG. 27 shows the observed denaturing temperature in the capillary chamber with a preset cycling temperature gradient ranging from 59-47° C. in 5 cycles. Due to the large difference between minimum and maximum temperature and taking into account the cycling speed, temperature within the capillary chamber did not reach the extreme preset temperatures during electrophoresis. Further the temperature at different locations in the capillary chamber was incongruent, especially at high denaturing temperatures. However, this discrepancy in microenvironment between capillaries with in the chamber did not have any significant effect on the separation (FIG. 26). The observed difference between maximum and minimum denaturing temperature within the capillary chamber was 8 K. As previously reported, fast cycling of the temperature may lead to narrowing of the temperature amplitude (Example 5), with loss of separation in some capillaries as a result. By applying large temperature gradients or temperature cycles during electrophoresis, the fragments are exposed to sub-optimal denaturing conditions for a longer time as compared to more focused denaturing conditions. Nevertheless, as long as the maximum denaturing temperature is below the denaturing temperature of the GC-clamp, separation of variants is preserved during electrophoresis (data not shown). By analyzing fragments at increasing temperature gradients, or increasing the average gradient temperature, specific “melting” characteristic of the low melting domain can be observed. Raking the peaks and subtracting the elution time of each species gives six different melting curves. FIG. 28 demonstrates the relative distance between peaks in fragment number 1 (BRCA1) at different denaturing temperatures. The optimal denaturing temperature of this fragment was calculated to be 48.6° C., a value that correspond well with separation maximum observed in FIG. 28. Separation of fragments at other denaturing temperature can also be expected as seen in the figure, and this quality allows compromising of the denaturing conditions in parallel analysis of fragments with different thermodynamics. Noteworthy is the melting of the heteroduplexes, which are, shifted a couple of degree lower, than homoduplexes, due to the miss-match in double stranded DNA (FIG. 28).

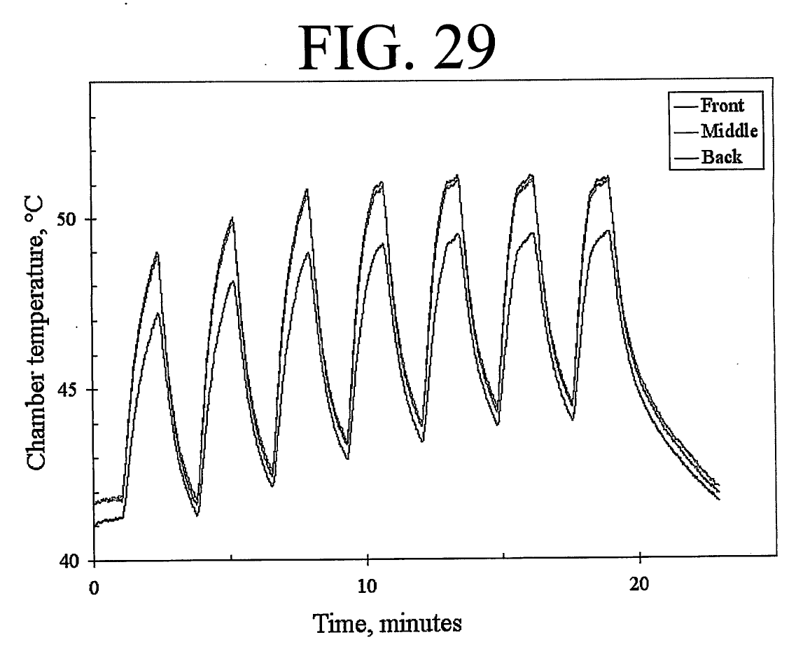

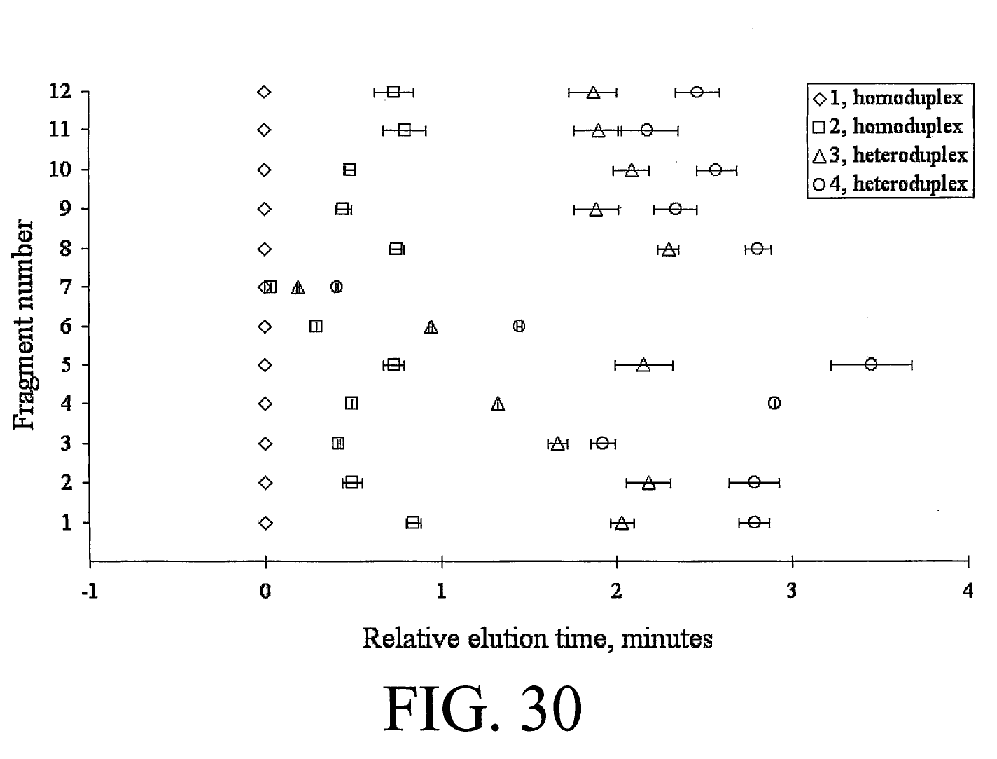

Cycling denaturing temperatures with smaller amplitudes was also investigated. Experiments demonstrated that with smaller denaturing cycles only fragments with theoretical melting temperature corresponding to the denaturing conditions separated during electrophoresis (data not shown). On the other hand, the separation between homoduplexes and heteroduplexes were better than with large cycling gradients. FIGS. 29 and 30 illustrates the observed denaturing temperature in the capillary chamber with a programmed gradient from 47 to 55° C., and the corresponding separation of fragments during electrophoresis, respectively. A large discrepancy between programmed and observed temperature was observed, when the start temperature was at the low extreme. This can be explained by the heat capacity of the instrument. With this setup the homoduplex in fragment 7 did not separate out, due to low maximum denaturing temperature. Nevertheless, separation of heteroduplexes was observed in all runs. The observed temperature during electrophoresis and the measured separation of alleles correspond well with the thermodynamics results described in FIG. 24.

Several analytical tools are designed for diagnostic analysis of DNA variation. One increasingly popular approach is DHPLC, which is based on similar separation principles, as the method reported here, were melting of double stranded DNA is separated in columns. By comparing the results reported herein and the literature one has to draw the conclusion that CTCE is superior to DHPLC by several means. The argument is described in the following paragraphs.

First, the cost of a DHPLC system is equivalent to a 48 capillary DNA sequencing system (MegaBACE 500 ™) were the obviously fact is that the CE instrument can analyze 48 samples at the time compared to one in the dHPLC system, when using only one channel of the DNA sequencing instrument. Acknowledging that DNA sequencing instruments in general have four channels, the sample throughput becomes hundred fold, that of the “high throughput” dHPLC (xiao, W. and Oefner, P., 2001, Hum. Mutat., 17:439-474). Furthermore, previously reported sample throughput of CTCE is limited to approximately 12000 samples per 24 hours in a 96 capillary instrument (Example 1). This is in gross contrast to the alleged high throughput of dHPLC, with a capacity of 144 samples in 24 hours.

Second, by implementing this assay one a regular DNA sequencing instrument, three application can be run on one instrument (DNA sequencing, fragment analysis and DNA variations assay) while only one assay, detection of DNA variation, can be run on dHPLC. In addition, given that DNA variations are detected by dHPLC a need for a second instrumentation (i.e., DNA sequencing instrument), or other means are needed to determine the type and position of the DNA variant (Hoogendoorn, B. et al., 1999, Hum. Genet., 104:89-93).

Third, the sensitivity defined as ratio of mutant versus wild type is reported to be in the 5 to 10% range for dHPLC (Wolford, J. et al., 2000, Hum. Genet., 107:483-487), while denaturant capillary electrophoresis has been reported down to 0.1% (Li-Sucholeiki, X. and Thilly, W., 2000, Nucleic Acids Res., 28:E44; Khrapko, K. et al., 2001, Methods Mol. Biol., 163:57-72). Consequently when analyzing heterogeneously sample like tumor tissue or pooled DNA, low mutant fraction cannot be detected by dHPLC.

Fourth, as demonstrated herein implementing firm knowledge of DNA thermodynamics and applying denaturing cycling gradient thereafter, any variation within the fragment can be separated on heteroduplex level and a large fraction on the homoduplex level. While dHPLC has to fine-tune the denaturing temperature for each variation to be analyzed, whether it is in the same fragment or in a different amplicon.

Last, there are no reports on separation of homozygote variants by dHPLC; save for large deletion on one allele, hence homozygote mutation or polymorphisms will not be detected. On the contrary, CTCE have clearly demonstrated baseline separation between homoduplexes and heteroduplexes in the results shown herein and in other reports (Example 2; Bjørheim, J. et al., 2003, Mutat. Res., 526:75-83; Kristensen, A. et al., 2002, BioTechniques, 33:650-653). Clearly the arguments above render CTCE a superior with regard to cost, sample throughput, specificity, sensitively and simplicity (one gradient fits all) as compared to dHPLC.

The analytical tool reported herein allows parallel analysis of different PCR products with up to 9 K difference in theoretical thermodynamics by CTCE. Ninety-six fragments are analyzed within 40 minutes, and sequence variants can be directly identified when control samples are available. If PCR primers are designed with almost identical Tₘ, different DNA sequences can be amplified simultaneously in 96-well plates, and the plates can thereafter be directly transferred to CTCE analysis. Since the fragments are exposed to cycling denaturing temperature during electrophoresis, new series (e.g., new 96-well plates) of fragments can be repeatedly be injected in the capillaries columns as previously reported (Example 1). The sample throughput of the method in diagnostic analysis is therefore, for any practical reasons, beyond obtainable number of patient samples in most settings. Further, different DNA sequences can be included in designed diagnostic 96-plates, relevant for different clinical settings and hypotheses, making the method amenable for a huge research community. Finally, the approach can easily be adapted to a fully automated set-up with the Caddy™—robotic sample plate manipulator used in conjunction with the MegaBACE™, which allows continuous analysis of samples without need of an operator.

### EXAMPLE 7

### Analysis of DNA Variation by Denaturant Capillary Electrophoresis

For all of the applications described below, alleles (DNA variants) are amplified in the same polymerase chain reaction (PCR) reaction by the same primer pair, subsequently followed by allele separation by electrophoresis.

PCR

To efficiently analyze DNA, the number of copies in a sample first was increased. This is performed through the well-characterized polymerase chain reaction. PCR does not only increase number of copies of DNA exponentially, but also restricts the amplification to specific target sequences determined by the primers. Specific PCR protocols are common in the art.

Electrophoresis

Capillary Electrophoresis (CE) was first described in 1967. During CE, a sample is separated into its components as it migrates through a capillary under the driving force of an electric field. Separation is typically performed in a long (10 to 100 cm), narrow (10 to 100 μm), electrolyte-filled, fused silica capillary. In the CE systems used to study DNA variants, a sieving matrix, e.g., chains of polymer, are used as separation media.

DNA Melting Gel Analysis

Theory of DNA Thermodynamics

In 1974, Poland proposed an algorithm able to calculate the melting probability of thousands of nucleotides in dsDNA (see above). Based on nearest neighbor correlation in specific sequence macromolecules and basic principles of DNA thermodynamics, the algorithm stated that dsDNA melts to single stranded DNA (ssDNA) when exposed to sufficiently high temperatures and/or chemical denaturants (e.g., formamide and urea). Specifically, the length of the DNA fragment and the nucleotide sequence within the fragment defines the melting temperature at which each base pair (bp) of a DNA duplex is in perfect equilibrium between the denatured and helical state. Importantly, DNA variants differing by only one base will reveal different melting properties based on the sequence variation. Differences in thermodynamics between differing DNA sequence such as base substitutions are exploited in melting gel techniques.

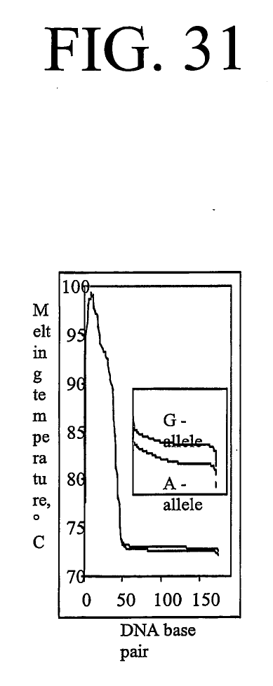

The need for approximations may rely on the fact that an exact algorithm required computer time proportional to N2, were N equals the number of bp in the target dsDNA. Computer software programs such as SQHTX, Melt87, WinMelt and the Poland internet web site calculate the melting profile of defined homozygous dsDNA fragments based on different related algorithms. Typically, stretches of double-stranded DNA consist of iso-melting domains with local thermodynamics covering a few hundred bases. The part of a fragment that melts at low denaturant exposure is known as the low melting domain, whereas the part that melts at high denaturant exposure is called the high melting domain (FIG. 31). The term “melting” refers to the change in the structure of DNA from an orderly helix to a disordered structure without base paring. The programs calculate the midpoint temperature at which each bp is at 50/50 equilibrium between the helical and melted states. Since the target sequence must be in the low melting domain, not all fragments will reveal a melting profile directly suitable for melting gel analysis. If the target sequence is part of a GC-rich area, it will often be part of a high melting domain. The goal of melting profile analysis of DNA fragments with computer programs is to select and manipulate the target sequence so that the region of interest is in the low melting domain. This often requires the proper placement of an artificial high melting domain. For a detailed description of fragment construction and GC-clamp attachment see (Example 5; Bjørheim, J. et al., 2003, Mutat. Res., 526:75-83). In theory, almost any DNA sequence can be analyzed with melting gel techniques as long as the base change results in sufficient differences in thermodynamics between alleles and that the target sequence is located in a low melting temperature domain adjacent to a high melting temperature domain (FIG. 31).

Denaturant Gradient Gel Electrophoresis and Related Techniques

Different melting gel techniques are named after the combination of denaturants in the gel (slab-gels) or matrix (capillary platform) and the platform on which the analysis is performed. Denaturant gradient gel electrophoresis (DGGE) has been the most frequently used melting gel techniques and was first described by Fisher and Lerman. DGGE has successfully been applied to detect mutations in genes including PKU, TP53, KRAS, NRAS, HRAS as well as genes involved in cystic fibrosis (Fischer, S. and Lerman, L., 1980, Proc. Natl. Acad. Sci. USA, 77:4420-4424; Eiken, H. et al., 1996, Hum. Mutat., 8:19; Nedergaard, T. et al., 1997, Int. J Cancer, 71:364; Wang, J. et al., 2000, Mol. Genet. Metab., 70:316). All other melting techniques are variants of this method. During Constant Denaturant Gel Electrophoresis (CDGE) the fragments are subjected to constant denaturing conditions in order to achieve better separation between alleles. CDGE has been used to detect base pair changes in defined target sequences such as TP53, BRCA1, RB1, hMSH2, MSH6, RET and HPRT (Smith-Sørensen, B. et al., 1992, Mutat. Res., 269:41; Børresen, A. et al., 1991, Proc. Natl. Acad. Sci. USA, 88:8405; Hovig, E. et al., 1992, Genes Chromosomes. Cancer, 5:97; Børresen, A. et al., 1995, Hum. Mol. Genet., 4:2065). On the other hand, the use of CDGE regulars careful casting of denaturing gels and control of denaturing temperature. If the denaturing conditions are slightly off scale separation will be lost. Other modifications of DGGE have focused on changing the temperature surrounding the gel and have resulted in methods including Temporal Temperature Gel Electrophoresis (TTGE; Yoshino, K. et al., 1991, Nucleic Acids Res., 19:3153) and Temperature Gradient Gel Electrophoresis (TGGE; Riesner, D. et al., 1989, Electrophoresis, 10:377). The denaturing conditions are therefore gradually changed during electrophoresis. The two main advantages of TTGE and TGGE compared with DGGE and CDGE are: 1) The simpler acrylamide gel casting procedure and 2) the potential to analyze fragments containing more than one isomelting domain. Both TTGE and TGGE have been employed in the analysis of KRAS, PTEN, mitochondrial DNA, TP53 and HLA. All of the above mentioned slab-gel methods have to some extent been extended to the capillary format.

Denaturant Capillary Electrophoresis

The modification of DGGE to the capillary platform has reduced the separation time of mutants, increased separation efficiency, and enabled the detection of low-frequency mutations in human cells and tissues with mutant fraction down to 10⁻⁶.

The application of Constant Denaturant Capillary Electrophoresis (CDCE) was developed on a laboratory assembled instrument and later adapted to commercial available capillary DNA sequencing instruments. In CDCE the optimal denaturing conditions for separation of DNA variants are determined by fine-tuning the temperature by re-analyzing the same sample at different temperatures. Within a certain temperature range, the position of the melting equilibrium and thus the average electrophoresis mobility of each mutant is different, which allows sequences containing single base pair point mutations to be separated. The CDCE method has been applied to analysis of DNA variation of several fragments including mitochondrial DNA APC, HRAS, KRAS and TP53. A further improvement of CDCE by modifying the instrumentation to allow two-point detection and automated fraction collection has demonstrated detection of mutations in samples with mutant fractions as low as 10⁻⁴. Despite the sensitivity and specificity of the method, few laboratories have applied this technique. This may rest upon the fact that polymer replacement and sample injection is performed manually, so the analysis is labor intensive and dependent on an instrument operator. Conversion of CDCE onto regular capillary DNA sequencing instruments has resulted in automation and standardized protocols. Compared to conventional CDCE in which each sample has to be loaded manually, automated CDCE analysis can be performed in a single or multi capillary instrument. The automation of CDCE allows for rapid analysis of a large number of samples over a short period of time and virtually any capillary sequencing instrument with a temperature controller can be used. The method was first described with use of an ABI 310 Genetic Analyser, in which up to 48 samples can be analyzed by this method without any need for operator intervention. The method has also been adapted to a regular 96-capillary DNA sequencing instrument, the MegaBACE™ 1000, allowing for analysis of 96 within 40 minutes.

The introduction of a temporal temperature gradient within the capillary was produced by a voltage ramp during electrophoresis. In this technique the temperature increase within the capillary due to the Joule heating during electrophoresis results in separation of alleles (Gelfi, C. et al., 1996, BioTechniques, 21:926-932). A different approach was later taken by modifying the CE system with a thermostatic liquid surrounding the capillary to allow for computer generated temporal temperature profiles between 25° C. and 70° C. A temperature gradient was also used in an automated 96-capillary DNA sequencing instrument to discriminate between mutations. Samples were analyzed with a temperature gradient, starting above and declining beneath the optimal separation temperature, by control of the instrument software. This technique proved to be robust in that the gradient compensated for the temperature differences within the capillary chamber, moreover each capillary passed through the optimal separation temperature around the theoretical melting temperature for the analyzed fragment. There was no need for time-consuming optimization procedures once the gradient has been established around the theoretical melting temperature of the fragment. Further improvement of the gradient concept was accomplished by cycling the gradient several times around the optimal separation temperature. This is based on the observation that during the cycling gradient, each capillary will pass through its optimal melting condition for the fragment of interest several times. This method is more robust than the CDCE when applied to multi-capillary instruments due to temperature differences within the capillary chamber.

Mutation Analysis

Detection of genetic changes is of central importance in molecular genetics and cancer research since alteration of just a single base pair in a DNA sequence can lead to change in cellular behavior. Knowledge of genotype markers, related to tumor behavior and patient prognosis, may lead to more effective preventive measures, prediction of prognosis and better personal medical treatment. Furthermore, the possibility of detecting mutations in a low fraction can be important for early detection of malignant diseases, detection of remaining cells after surgery and possibly for prognosis and outcome of the disease.

In two reports, denaturant capillary electrophoresis (DCE) was applied to the detection of mutations in KRAS exon 1 and exons 5-8 of TP53 gene by use of an ABI 310 Genetic Analyzer, with a sensitivity (mutant/wild-type) down to 10⁻³ (Bjørheim, J. et al., 2001, Tumour. Biol., 22:323-327; Bjørheim, J. et al., 1998, Mutat.Res., 403:103). The same application was also demonstrated on a modified 96-capillary DNA sequencing instrument, the MegaBACE™1000. The need for modification of the instrument rested upon the need for accurate temperature control in all 96 capillaries, so an external heater was fitted to the capillary array. To circumvent the use of an external heater a temperature gradient controlled by the instrument software was later applied to the sequencer.

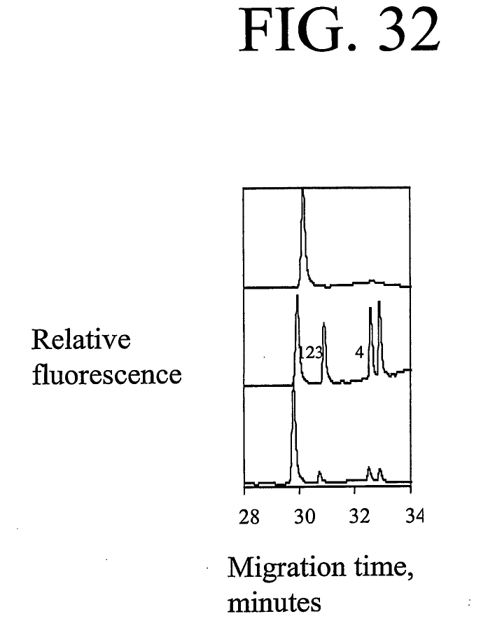

This modified technique was used to successfully separate mutants in exon 8 of the TP53 gene in all 96 capillaries (Kristensen, A. et al., 2001, BioTechniques, 33:650-654). In a reconstruction experiment the sensitivity to detect mutations alleles in a wild type background was 0.4 %. To further improve the reproducibility of the separation in a multi capillary instrument, the element of cycling temperature during electrophoresis was introduced. By changing the temperature around the optimal separation temperature, almost equal denaturing conditions in all capillaries and between runs was obtained. As a result, highly reproducible denaturing conditions were observed, which resulted in detection and identification of all twelve different KRAS exon 1 mutations (Example 2). Furthermore, we have applied DCE (CDCE, TGCE and CTCE) analysis of mutations in several genes (KRAS, TP53, NRAS, PTEN and BRAF). The figure below demonstrates detection of mutants in samples with different mutant fraction, by DCE (FIG. 32).

Single Nucleotide Polymorphisms (SNP)

A SNP is defined as base substitution with a frequency above 1% in the population of interest. When two randomly chosen alleles are compared, sequence variations are expected to be found every 1000 bp. Consequently, one would expect many sequence differences when analyzing larger populations as compared to the canonical sequence. In addition, SNPs are typically bi-allelic and are therefore becoming popular to discriminate between alleles or haplotypes in medical and population genetic studies. Some of these sequence variants may affect functions such as immune response, metabolism, DNA repair, or control of cell death and division. Finally, some variants may be regarded as SNP in one population and as a mutation in another cohort.

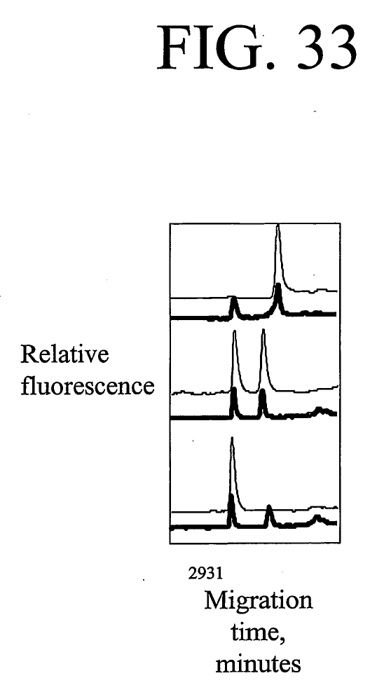

An approach utilizing DCE for SNP analysis is described herein. Target sequences were PCR amplified followed by allele separation by capillary electrophoresis. The genotypes of the individuals SNPs were scored based on co-migration to an internal standard. The fragment specific internal standard was made from a sample with both variants (alleles) of the SNP and was labeled with a different fluorophore than the sample (FIG. 33).

Due to the presence of the internal standard in all the electrophoretic runs, the specificity of the SNP analysis is retained at an excellent level because allele separation of the standard is required before genotypes can be determined.

Microhaplotypes

A Microhaplotypes is in general defined as the presence of several SNPs within a short fragment of DNA (100-1000 bp). The theoretical number of possible alleles is defined as 2 n, were n is number of SNPs in the sequence analyzed. Possible combinations of genotypes are given by the sum of numbers from zero up to numbers of alleles (e.g., 4=alleles, genotypes=4+3+2+1). Few methods are able to analyze microhaplotypes, and the protocols are in general labor intensive and not easily automated. Consequently, DCE has been optimized for microhaplotyping on some fragments by use of standard DCE protocol, e.g., by PCR and electrophoresis. DCE was used to identify multiple SNPs within a defined target sequence based on theoretical thermodynamics and migration of variant fragments (Krisetensen, A. et al., in press). This target sequence is located in the Ataxia-telangiectasia (ATM) gene, in which three SNPs are reported.

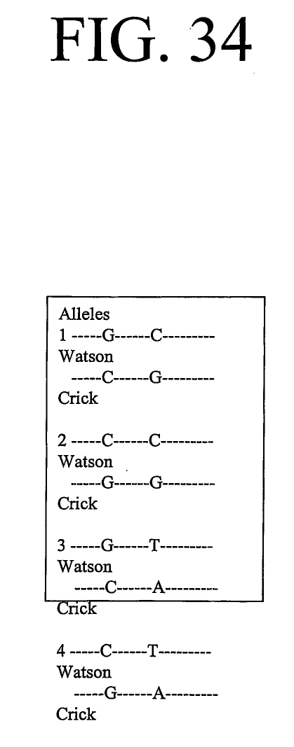

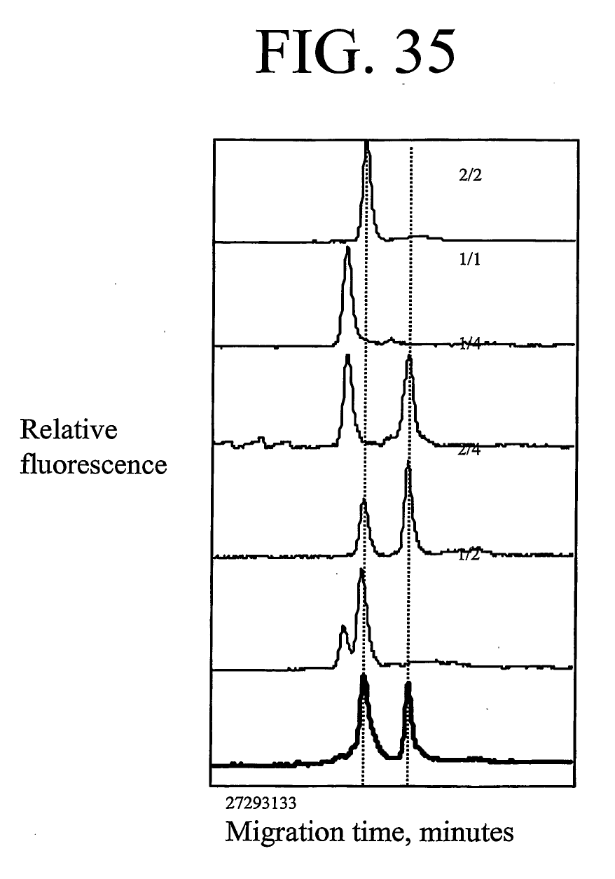

Fluorophore-labeled PCR products were analyzed without any post PCR steps on a standard 96 capillary-sequencing instrument MegaBACE™1000. 3677 samples were analyzed for microhaplotypes, to validate the method, and further to establish microhaplotype frequencies in the target populations. This direct SNP detection and haplotype determination based on co-elution have added one more level of information to the SNP analysis. In an ongoing study of familial breast cancer, microhaplotyping is used to determine haplotype sharing in BRCA 1 and 2 genes and has efficiently determined microhaplotypes in several thousand individuals. Exemplified microhaplotypes from two adjacent SNPs located on chromosome 18q21.1 are shown in FIG. 34. Alleles were separated by DCE in a standard DNA capillary sequencing instrument and genotyped according to an internal standard having allele 2 and 4 (FIG. 34). Electropherograms of selected samples are presented in FIG. 35.

With only one PCR reaction and one electrophoresis two neighboring SNPs can be determined with the additional information of microhaplotype. The application has therefore been improved from labor intensive and low capacity to a straightforward and high-throughput method for microhaplotype determination.

Gene Copy Number

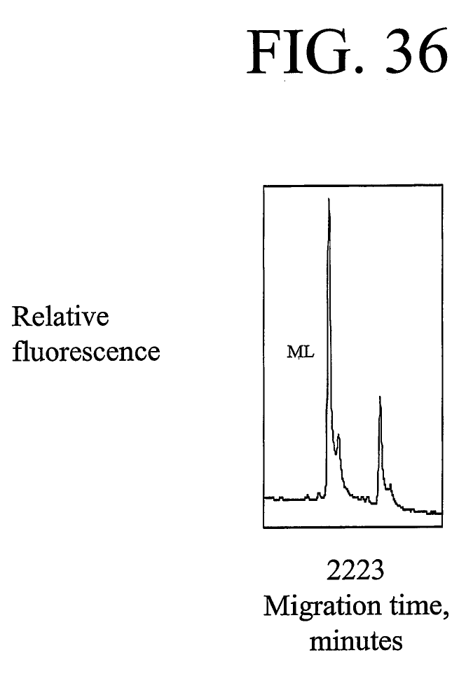

Pooling of samples is an obvious approach when analyzing SNPs in large populations. Described above is a method where the population frequency of SNP can be efficiently detected, and their allele frequencies accurately measured (Example 3). In this example, 8000 alleles were analyzed for selected SNPs. There was no need for any correction of the signal of the separated alleles because both alleles are labeled with the same fluorophore and because fragments of the same length with the differences of one base pair will pass the detector with the same velocity. Thus, quantum yield will reflect DNA copies entered in the PCR. Additionally, no preferential amplification of alleles could be observed when areas under the peaks were measured in heterozygous individual samples. The ratio of alleles was essentially 1:1. The result of the study gave good correspondence between estimated allele frequencies in pooled samples and genotypes verified by analysis of the single samples. A similar approach applied to a different application is to analyze the Opsin color vision gene array. Within this gene there are several copies on the same chromosome. The tandem array is believed to consist of a single red (L) gene, and one to seven copies of the green (M) gene, as a result of uneven intergenic crossing-over. The red and green genes differ by fifteen amino acid residues, and 7 of these 15 are responsible for the absorption spectra. Quantification of the gene ratio is demonstrated in FIG. 36. By measuring the area under the peaks (alleles), quantitative information about DNA copies entering the PCR reaction is obtained.

The same concept has also been applied for the analysis of expression differences at mRNA level. By use of heterozygous intra exonic SNPs differences in mRNA expression can be observed and would give similar electropherogram as in FIG. 36.

Allelic Imbalance

Study of human cancers has indicated a variety of inactivation of multiple tumor suppressor genes for tumor development and/or progression cancer. Loss of genes or its function can occur through mutation of one allele together with the loss of the second allele, or through homozygous deletion of the gene. To determine allelic imbalance (allelic loss/reduction or allelic gain) in tumor samples, use of polymorphic microsatellite markers at gene loci of interest has traditionally been used. A disadvantage of using microsatellites is that they are rare (compared to SNPs) and generally located in non-coding regions of the genome. To circumvent the use of microsatellites we have applied regular SNP analysis by DCE as a means for determination allelic imbalance.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for separating nucleic acids comprising electrophoresing a sample applied to a gel electrophoresis matrix in a capillary, wherein during electrophoresis, the temperature of the matrix is cycled at least two times between a high and low temperature.

2. The method of claim 1, wherein the nucleic acids to be separated are DNA fragments comprising one or more polymorphic sites.

3. The method of claim 2, wherein allelic variants at the one or more polymorphic sites are separated.

4. The method of claim 1, wherein the temperature is initially at a high temperature and the first cycle is from a high temperature to a low temperature.

5. The method of claim 1, wherein the high temperature and/or low temperature is different during successive cycles.

6. The method of claim 1, wherein the temperature is cycled from about 2 to 60 times.

7. The method of claim 4, wherein the temperature is cycled about 20 times.

8. The method of claim 1, wherein the high temperature is about 3° C. higher than the low temperature.

9. The method of claim 1, wherein the temperature is between about 2° C. and about 15° C. higher than the lower temperature.

10. The method of claim 1, wherein the higher temperature is between about 3° C. and about 10° C. higher than the lower temperature.

11. The method of claim 1, wherein the high temperature is less than about 80° C.

12. The method of claim 1, wherein the low temperature is about 40° C.

13. The method of claim 1, wherein the high temperature is between 50° C. and 75° C.

14. The method of claim 1, wherein the low temperature is between 40° C. and 50° C.

15. The method of claim 1, further comprising detecting dsDNA after electrophoresis.

16. The method of claim 13, wherein, after the desired number of temperature cycles have been completed, the temperature of the gel matrix is such that DNA remains double-stranded.

17. The method of claim 1, wherein the temperature oscillations are ramped to provide optimal separation of the alleles.

18. A method for estimating allele frequency comprising:
electrophoresing a sample applied to a capillary gel electrophoresis matrix, wherein during electrophoresis, the temperature of the matrix is cycled at least two times, wherein one cycle is from a high temperature to a low temperature or from a low temperature to a high temperature, thereby separating DNA molecules in the sample; and
quantifying the variant sequences of the separated DNA molecules
thereby providing an estimate of the allele frequency for each variant DNA molecule.

19. The method of claim 18, further comprising detecting dsDNA after electrophoresis.

20. The method of claim 19, wherein, after the desired number of temperature cycles have been completed, the temperature of the gel matrix is such that DNA remains double-stranded.

21. A method for detecting a microhaplotype comprising separating DNA fragments comprising a sequence comprising two or more polymorphic sites of the microhaplotype, wherein the fragments are separated by capillary electrophoresis performed with two or more temperature oscillations between a high and a low temperature.

22. The method of claim 21, wherein the temperature oscillations are ramped to provide optimal separation of the microhaplotype.
